(19) United States
(12) Patent Application Publication
(10) Pub. No.: US 2019/0015453 A1
(43) Pub. Date: Jan. 17, 2019

(54) MicroRNAS FOR THE GENERATION OF ASTROCYTES

(71) Applicants: EXOSTEM BIOTEC LTD., Tel Aviv (IL); HENRY FORD HEALTH SYSTEM, Detroit, MI (US)

(72) Inventors: Chaya BRODIE, Southfield, MI (US); Aharon BRODIE, Miami Beach, FL (US)

(21) Appl. No.: 16/048,676

(22) Filed: Jul. 30, 2018

Related U.S. Application Data
(63) Continuation-in-part of application No. 14/380,155, filed on Aug. 21, 2014, now Pat. No. 10,034,902.
(60) Provisional application No. 61/601,624, filed on Feb. 22, 2012.

(51) Int. Cl. A61K 35/28; C12N 5/0775; C12N 5/079; A61P 25/16

(52) U.S. Cl. CPC A61K 35/28; C12N 5/0662; C12N 5/0622; C12N 2501/65; C12N 2510/00; C12N 2506/1346; A61P 25/16

(57) ABSTRACT

A method of generating a population of cells useful for treating a nerve disease or disorder in a subject, the method comprising up-regulating a level of at least one exogenous miRNA in mesenchymal stem cells (MSCs) and/or down-regulating a level of at least one miRNA using a polynucleotide agent that hybridizes to the miRNA, thereby generating the population of cells useful for treating the nerve disease or disorder. Isolated populations of cells with an astrocytic phenotype generated thereby and uses thereof are also provided.

### CROSS REFERENCE TO RELATED APPLICATIONS

This application is a Continuation-in-Part of U.S. patent application Ser. No. 14/380,155 filed Aug. 21, 2014 which is a 371 (c)(1) National Phase entry of International Patent Application No. PCT/IB13/051430 filed on Feb. 21, 2013, which claims the benefit of priority of U.S. Provisional Patent Application No. 61/601,624, filed Feb. 22, 2012. The contents of the above applications are all hereby expressly incorporated by reference, in their entirety.

### FIELD AND BACKGROUND OF THE INVENTION

The present invention, in some embodiments thereof, relates to methods of ex vivo differentiating mesenchymal stem cells towards astrocytic cells using microRNAs.

Mesenchymal stem cells (MSCs) are a heterogeneous population of stromal cells that can be isolated from multiple species, residing in most connective tissues including bone marrow, adipose, placenta, umbilical cord and perivascular tissues. MSCs can also be isolated from the placenta and cord's Wharton's jelly.

The concentration of MSCs in all tissues, including bone marrow and adipose tissue is very low but their number can be expanded in vitro. Typically, expansion of MSCs using up to 15 passages does not result in mutations indicating genetic stability.

MSC can differentiate into cells of the mesenchymal lineage, such as bone, cartilage and fat but, under certain conditions, have been reported to acquire the phenotype of cells of the endodermal and neuroectodermal lineage, suggesting some potential for “transdifferentiation”.

Within the bone marrow compartment, these cells are tightly intermingled with and support hematopoiesis and the survival of hematopoietic stem cells in acquiescent state (7). In addition, after expansion in culture, MSCs retain their ability to modulate innate and adaptive immunity (8). Furthermore, MSCs migrate actively to sites of inflammation and protect damaged tissues, including the CNS, properties that supported their use as new immunosuppressive or rather immunoregulatory or anti-inflammatory agents for the treatment of inflammatory and immune-mediated diseases including autoimmune disorders (9). These features of MSCs merited their use to control life-threatening graft-versus-host-disease (GVHD) following allogeneic bone marrow transplantation, thus controlling one of the most serious complications of allogeneic bone marrow transplantation, helping to lower transplant-related toxicity and mortality associated with multi-system organ injury (10).

Several studies have shown that MSCs following exposure to different factors in vitro, change their phenotype and demonstrate neuronal and glial markers [Kopen, G. C., et al., Proc Natl Acad USA. 96(19):10711-6, 1999; Sanchez-Ramos, et al. Exp Neurol. 164(2): 247-56. 2000; Woodbury, D., J Neurosci Res. 61(4): 364-70, 2000; Woodbury, D., et al., J Neurosci Res. 69(6):908-17, 2002; Black, I. B., Woodbury, D. Blood Cells Mol Dis. 27(3):632-6, 2001; Kohyama, J., et al. Differentiation. 68(4-5):235-44, 2001; Levy, Y. S. J Mol Neurosci. 21(2):121-32, 2003].

Accordingly, MSCs (both ex-vivo differentiated and non-differentiated) have been proposed as candidates for cell replacement therapy for the treatment of various neurological disorders including multiple sclerosis, Parkinson's disease, ALS, Alzheimer's disease, spinal

As an alternative to neuronal cell replacement strategy, in order to increase the survival of existing functional and morphologically normal cells, cell therapy may be aimed at restoring or reestablishing the normal anatomy (e.g. connectivity) and physiology (e.g. appropriate synaptic contacts and functioning neurotransmitters and neuroregulators) of a diseased or damaged tissue.

Astrocytes are the most abundant type of glial cells in the central nervous system and play major roles in the development and normal physiological functions of the brain. Mature astrocytes are divided into two types: fibrous and protoplasmic astrocytes.

Fibrous astrocytes populate the white matter and typically have a ‘star-like’ appearance with dense glial filaments that can be stained with the intermediate filament marker glial fibrillary acidic protein (GFAP). Protoplasmic astrocytes are found in the grey matter, have more irregular, ‘bushy’, processes and typically have few glial filaments. These cells come into contact with and ensheath thin processes, some of which also contact blood vessels.

Astrocytes also regulate water balance, redox potential and ion and neurotransmitter concentrations, secrete neurotrophic factors, remove toxins and debris from the cerebrospinal fluid (CSF) and maintain the blood-brain bather. They also participate in cell-cell signaling by regulating calcium flux, releasing d-serine, producing neuropeptides and modulating synaptic transmission.

Since astrocytes provide structural and physiological support in the central nervous system, differentiation of MSCs towards an astrocytic lineage has been proposed for the treatment of neurological disorders.

Various cells type produce GDNF including glia cells (oligodendrocytes and astrocytes), neuroblastoma and glioblastoma cell lines. It has been shown that rat BMSCs cultured in DMEM supplemented with 20% fetal bovine serum, at passage 6 express GDNF and NGF [Garcia R, et al., Biochem Biophys Res Commun. 316(3):753-4, 2004].

International Patent Publications WO2006/134602 and WO2009/144718 teach differentiation of mesenchymal stem cells into cells which produce neurotrophic factors.

International Patent Publication WO2010/111522 teaches mesenchymal stem cells which secrete and deliver microRNAs for the treatment of diseases.

International Patent Publication WO2010/144698 teaches expression of miRNAs in

International Application No. IL2011/000660 teaches expression of miRNAs in mesenchymal stem cells to induce oligodendrocytic differentiation thereof.

### SUMMARY OF THE INVENTION

According to an aspect of some embodiments of the present invention there is provided a method of generating a population of cells useful for treating a nerve disease or disorder in a subject, the method comprising up-regulating a level of at least one exogenous miRNA being selected from the group consisting of miR-1293, miR-18, miR-1182, miR-1185, miR-1276, miR-17-5p, miR-141, miR-302b, miR-20b, miR-101, miR-126, miR-146a, miR-146b, miR-3a, miR-29, miR-504, miR-891, miR-874 and miR-132 in mesenchymal stem cells (MSCs), thereby generating the population of cells useful for treating the nerve disease or disorder.

According to an aspect of some embodiments of the present invention there is provided a method of generating a population of cells useful for treating a nerve disease or disorder in a subject, the method comprising down-regulating an expression of at least one miRNA, the miRNA being selected from the group consisting of mi-R-193b, mi-R-1238, miR-889, mi-R-370, mi-R-548-d1, mi-R-221, mi-R-135a, mi-R-149, mi-R-222, mi-R-199a, mi-R-302a, miR-302b, mi-R-302c, mi-R-302d, mi-R-369-3p, mi-R-let7a, mi-R-let7b, mi-R-10b, mi-R-23a, mi-R-23b, mi-R-138, mi-R-182, mi-R-487, mi-R-214, mi-R-409, miR-133, miR-145 and mi-R-32, wherein the down-regulating is effected by up-regulating a level of at least one polynucleotide agent that hybridizes and inhibits a function of the at least one miRNA thereby generating the population of cells useful for treating the nerve disease or disorder.

According to an aspect of some embodiments of the present invention there is provided a method of generating a population of cells useful for treating a nerve disease or disorder in a subject, the method comprising up-regulating a level of exogenous miR-9 and exogenous miR-20b in a population of MSCs, thereby generating the population of cells.

According to an aspect of some embodiments of the present invention there is provided a method of generating a population of cells useful for treating a nerve disease or disorder in a subject, the method comprising up-regulating a level of exogenous miR-9, exogenous miR-146 and exogenous miR-101 in a population of MSCs and down-regulating an expression of miR-10b and miR-302 using in the population of MSCs thereby generating the population of cells.

According to an aspect of some embodiments of the present invention there is provided a method of generating a population of cells useful for treating a nerve disease or disorder in a subject, the method comprising up-regulating a level of exogenous miR-101 in a population of MSCs and down-regulating an expression of miR-138 in the population of MSCs thereby generating the population of cells.

According to an aspect of some embodiments of the present invention there is provided a genetically modified isolated population of cells which comprise at least one exogenous miRNA selected from the group consisting of miR-18, miR-17-5p, miR-141, miR-302b, miR-20b, miR-101, miR-126, miR-146a, miR-146b, miR-9, miR-504, miR-891, miR-874, miR-1182, miR-1185, miR-1276, miR-1293 and miR-132 and/or at least one polynucleotide agent that hybridizes and inhibits a function of a miRNA being selected from the group consisting of mi-R-193b, mi-R-221, mi-R-135a, mi-R-149, mi-R-222, mi-R-199a, mi-R-302a, mi-R-302c, mi-R-302d, mi-R-369-3p, mi-R-370, mi-R-let7a, mi-R-let7b, mi-R-10b, mi-R-23a, mi-R-23b, mi-R-138, mi-R-182, mi-R-487, mi-R-214, mi-R-409, mi-R-548-d1, mi-R-889, mi-R-1238 and mi-R-32, the cells having an astrocytic phenotype.

According to an aspect of some embodiments of the present invention there is provided a method of treating a nerve disease or disorder in a subject in need thereof, the method comprising administering to the subject a therapeutically effective amount of the isolated population of cells described herein, thereby treating the nerve disease or disorder.

According to an aspect of some embodiments of the present invention there is provided a pharmaceutical composition comprising the isolated population of cells described herein and a pharmaceutically acceptable carrier.

According to an aspect of some embodiments of the present invention there is provided a method of selecting a miRNA which may be regulated for the treatment of a nerve disease or disorder comprising:

(a) differentiating a population of MSCs towards an astrocytic phenotype; and

(b) analyzing a change in expression of a miRNA in the population of MSCs prior to and following the differentiating of the MSCs towards an astrocytic phenotype, wherein a change of expression of a miRNA above or below a predetermined level is indicative that the miRNA may be regulated for the treatment of the nerve disease or disorder.

According to an aspect of some embodiments of the present invention there is provided a method of generating a population of cells useful for treating a nerve disease or disorder in a subject, the method comprising up-regulating a level of at least one exogenous miRNA set forth in Table 1 in mesenchymal stem cells (MSCs), thereby generating the population of cells useful for treating the nerve disease or disorder.

According to an aspect of some embodiments of the present invention there is provided a method of generating a population of cells useful for treating a nerve disease or disorder in a subject, the method comprising down-regulating a level of at least one exogenous miRNA set forth in Table 2 in mesenchymal stem cells (MSCs), thereby generating the population of cells useful for treating the nerve disease or disorder.

According to an aspect of some embodiments of the present invention there is provided a method of treating Parkinson's disease in a subject in need thereof, comprising administering to the subject a therapeutically effective amount of MSCs which have been modified to express an exogenous miR504, thereby treating Parkinson's.

According to an aspect of some embodiments of the present invention there is provided a genetically modified isolated population of cells which comprise at least one exogenous miRNA selected from the group consisting of miR-18, miR-1293, miR-1182, miR-1185 and miR-1276 and/or at least one polynucleotide agent that hybridizes and inhibits a function of a miRNA being selected from the group consisting of mi-R-193b, mi-R-1238, miR-889, mi-R-370 and mi-R-548-d1, said cells having an astrocytic phenotype.

According to some embodiments of the invention, the at least one miRNA is selected from the group consisting of miR-18, miR-1293, miR-1182, miR-1185 and miR-1276.

According to some embodiments of the invention, the at least one miRNA is selected from the group consisting of miR-20b, miR-146, miR-101 and miR-141.

According to some embodiments of the invention, the at least one miRNA is selected from the group consisting of miR-32, miR-221, miR-302a and miR-302b.

According to some embodiments of the invention, the at least one miRNA is selected from the group consisting of mi-R-193b, mi-R-1238, miR-889, mi-R-370 and mi-R-548-d1.

According to some embodiments of the invention, the at least one miRNA comprises each of the miR-20b, the miR-101 and the miR-146a.

According to some embodiments of the invention, the MSCs are isolated from a tissue selected from the group consisting of bone marrow, adipose tissue, placenta, cord blood and umbilical cord.

According to some embodiments of the invention, the MSCs are autologous to the subject.

According to some embodiments of the invention, the MSCs are non-autologous to the subject.

According to some embodiments of the invention, the MSCs are semi-allogeneic to the subject.

According to some embodiments of the invention, the up-regulating comprises introducing into the MSCs the miRNAs.

According to some embodiments of the invention, the up-regulating is effected by transfecting the MSCs with an expression vector which comprises a polynucleotide sequence which encodes a pre-miRNA of the at least one miRNA.

According to some embodiments of the invention, the up-regulating is effected by transfecting the MSCs with an expression vector which comprises a polynucleotide sequence which encodes the at least one miRNA.

According to some embodiments of the invention, the method further comprises analyzing an expression of at least one marker selected from the group consisting of S100, GFAP, glutamine synthetase, EAAT1 and EAAT2 following the generating.

According to some embodiments of the invention, the method is effected in vivo.

According to some embodiments of the invention, the method is effected ex vivo.

According to some embodiments of the invention, the method further comprises incubating the MSCs in a differentiation medium comprising at least one agent selected from the group consisting of platelet derived growth factor (PDGF), neuregulin, FGF-b and a c-AMP inducing agent following, prior to or concomitant with the contacting.

According to some embodiments of the invention, at least 50% of the population of cells express at least one marker selected from the group consisting of S100, GFAP, glutamine synthetase, EAAT1 and EAAT2.

According to some embodiments of the invention, the isolated population of cells is for use in treating a brain disease or disorder.

According to some embodiments of the invention, the brain disease or disorder is a neurodegenerative disorder.

According to some embodiments of the invention, the neurodegenerative disorder is selected from the group consisting of multiple sclerosis, Parkinson's, epilepsy, amyotrophic lateral sclerosis (ALS), stroke, Rett Syndrome, autoimmune encephalomyelitis, stroke, Alzheimer's disease and Huntingdon's disease.

According to some embodiments of the invention, the nerve disease or disorder is a neurodegenerative disorder.

According to some embodiments of the invention, the neurodegenerative disorder is selected from the group consisting of multiple sclerosis, Parkinson's, epilepsy, amyotrophic lateral sclerosis (ALS), stroke, Rett Syndrome, autoimmune encephalomyelitis, stroke, Alzheimer's disease and Huntingdon's disease.

According to some embodiments of the invention, the method further comprises analyzing expression of an astrocyte specific gene following step (a) and prior to step (b).

According to some embodiments of the invention, the astrocyte specific gene is GFAP.

According to some embodiments of the invention, the neurodegenerative disorder is selected from the group consisting of multiple sclerosis, Parkinson's, epilepsy, amyotrophic lateral sclerosis (ALS), stroke, Rett Syndrome, autoimmune encephalomyelitis, stroke, Alzheimer's disease and Huntingdon's disease.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1F are photographs illustrating that MSCs may be differentiated into astrocyte-like cells. BM-MSCs were incubated with the differentiation media and were then analyzed for cell morphology using phase contrast microscopy and were stained with anti-GFAP antibody. Similar results were obtained with AD-MSCs and with MSCs derived from cord and from placenta (data not shown).

FIG. 2 is a bar graph illustrating that differentiated MSCs express astrocytic markers. Control and differentiated MSCs were treated as described in the methods. RNA was extracted, and qRT-PCR was performed using primers for GFAP, glutamine synthetase and S100.

FIG. 3 is a bar graph illustrating that differentiated MSCs express glutamate transporters. Control and differentiated MSCs were treated as described in the methods. RNA was extracted, and qRT-PCR was performed using primers for glutamate transporters.

FIG. 4 is a bar graph representing results of the analysis of miRNA signature of stem cell sets of miRNAs. This set consists of miRNAs that are associated with stem cell signature and self renewal.

FIG. 5 is a bar graph representing results of the analysis of miRNA signature of the neural set of miRNAs. This set consists of miRNAs that are associated with neural development.

FIG. 6 is a bar graph representing results of the analysis of miRNA signature of the hematopoietic set of miRNAs. This set consists of miRNAs that are associated with hematopoiesis.

FIG. 7 is a bar graph representing analysis of miRNA signature of the organ set of miRNAs. This set consists of miRNA that are associated with differentiated tissue identification.

FIG. 8 is a bar graph illustrating a change in expression of exemplary miRNAs during astrocytic differentiation of MSCs as measured by quantitative RT-PCR.

FIGS. 9A-9B are photographs of BM-MSCs transduced with a GFAP-GFP reporter. In FIG. 9B, the MSCs were transfected with both antagomiR-138 and miR-101. The cells were viewed under a fluorescence microscope after 10 days.

FIG. 10 is a photograph of results of a Western blot analysis illustrating that miRNA 504 downregulates a synuclein in SH-SY5Y cells (lane 1=control; lanes 2+3=miRNA 504).

FIG. 11 is a bar graph illustrating target validation of miR-504 and anti-miR-302. MSCs or their derived exosomes were co-cultured with SH-5Y cells in a transwell plate. MSCs were transfected with either a control miR, miR-504 or a combination of miR-504 and anti-miR-302, and SH-5Y were transfected with an alpha-synuclein 3′-UTR-luciferase reporter plasmid. The luciferase activity of these cells was measured 72 hours thereafter. The results represent the means±SD of three separate experiments.

FIG. 12 is a bar graph showing increased expression of a GFAP reporter tagged to GFP in MSCs when transfected with miR-504, anti-miR-302 or their combination. Values are (%) of GFAP positive cells after 3 days in culture.

FIGS. 13A-13B are bar graphs showing MSC differentiation. MSCs transfected with control miR, miR-504 or a combination of miR-504 and anti-miR-302 were shown to over express (13A) GDNF and (13B) the glutamate transporter EAAT2.

### DESCRIPTION OF SPECIFIC EMBODIMENTS OF THE INVENTION

The present invention, in some embodiments thereof, relates to methods of ex vivo differentiating mesenchymal stem cells towards astrocytic cells using microRNAs.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Astrocytes are the most abundant type of glial cells in the central nervous system and play major roles in the development and normal physiological functions of the brain. Mature astrocytes are divided into two types: fibrous and protoplasmic astrocytes.

Fibrous astrocytes populate the white matter and typically have a ‘star-like’ appearance with dense glial filaments that can be stained with the intermediate filament marker glial fibrillary acidic protein (GFAP). Protoplasmic astrocytes are found in the grey matter, have more irregular, ‘bushy’, processes and typically have few glial filaments. These cells come into contact with and ensheath of thin processes, some of which also contact blood vessels.

Astrocytes also regulate water balance, redox potential and ion and neurotransmitter concentrations, secrete neurotrophic factors, remove toxins and debris from the cerebrospinal fluid (CSF) and maintain the blood-brain bather. They also participate in cell-cell signaling by regulating calcium flux, releasing d-serine, producing neuropeptides and modulating synaptic transmission.

Since astrocytes provide structural and physiological support in the central nervous system, generation of cells which have an astrocytic phenotype has been proposed for the treatment of neurological disorders.

Whilst reducing the present invention to practice, the present inventors have found that out of a vast number of potential micro RNAs (miRNAs), only up-regulation of particular miRNAs including miR-18, miR-17-5p, miR-141, miR-302b, miR-20b, miR-101, miR-126, miR-146a, miR-146b, miR-3a, miR-26, miR-29, miR-504, miR-891, miR-874, miR-1182, miR-1185, miR-1276, miR-1293 and miR-132 induces astrocytic differentiation of mesenchymal stem cells (MSCs) and propose that such differentiated MSCs may be used to treat patients with brain diseases or disorders.

Specifically, the present inventors have shown that transfection of MSCs with particular combinations of the miRNAs listed above (e.g. the combination of miR-9 and miR-20b as well as the combination of miR-20b, 101 and 146a) changed the morphological appearance of the cells and further increased expression of various astrocytic markers therein (e.g. GFAP expression).

In addition, the present inventors have identified a number of miRNAs whose down-regulation is associated with astrocytic differentiation of MSCs. Included in this list are mi-R-193b, mi-R-221, mi-R-135a, mi-R-149, mi-R-222, mi-R-199a, mi-R-302a, mi-R-302c, mi-R-302d, mi-R-369-3p, mi-R-370, mi-R-let7a, mi-R-let7b, mi-R-10b, mi-R-23a, mi-R-23b, mi-R-32, miR-133, mi-R-145, mi-R-138, mi-R-182, mi-R-487, mi-R-214, mi-R-409, mi-R-548-d1, mi-R-889 and mi-R-1238. Further it was found that inhibiting miR-10b and miR-302 whilst at the same time over expressing miR-9, 146 and 101 enhanced differentiation towards an astrocytic phenotype as measured by GFAP expression. In addition, it was found that inhibiting miR-138, whilst at the same time overexpressing miR-101 enhanced differentiation towards an astrocytic phenotype as measured by GFAP expression.

Thus, according to one aspect of the present invention, there is provided a method of generating a population of cells useful for treating a nerve disease or disorder in a subject, the method comprising up-regulating a level of at least one exogenous miRNA being selected from the group consisting of miR-18, miR-17-5p, miR-141, miR-302b, miR-20b, miR-101, miR-126, miR-146a, miR-146b, miR-3a, miR-26, miR-29, miR-132, miR-504, miR-891, miR-874, miR-1182, miR-1185, miR-1276 and miR-1293 in mesenchymal stem cells (MSCs), thereby generating the population of cells useful for treating the nerve disease or disorder.

Additional miRNAs contemplated for upregulation are provided herein below. miR-92ap, miR-21, miR-26a, miR-18a, miR-124, miR-99a, miR-30c, miR-301a, miR-145-50, miR-143-3p, miR-373, miR-20b, miR-29c, miR-29b, miR-143, let-7g, let-7a, let-7b, miR-98, miR-30a*, miR-17, miR-1, miR-192, miR-155, miR-516-ap, miR-31, miR-181a, miR-181b, miR-181c, miR-34-c, miR-34b*, miR-103a, miR-210, miR-16, miR-30a, miR-31, miR-222, miR-17, miR-17*, miR-200b, miR-200c, miR-128, miR-503, miR-424, miR-195, miR-1256, miR-203a, miR-199, miR-93, miR-98, miR-125-a, miR-133a, miR-133b, miR-126, miR-194, miR-346, miR-15b, miR-338-3p, miR-373, miR-205, miR-210, miR-125, miR-1226, miR-708, miR-449, miR-422, miR-340, miR-605, miR-522, miR-663, miR-130a, miR-130b, miR-942, miR-572, miR-520, miR-639, miR-654, miR-519, mir-202, mir-767-5p, mir-29a, mir-29b, mir-29c, let-7a, let-7b, let-7c, let-7d, let-7e, let-7f, let-7g, let-7i, mir-4458, mir-4500, mir-98, mir-148a, mir-148b, mir-152, mir-4658, mir-3662, mir-25, mir-32, mir-363, mir-367, mir-92a, mir-92b, mir-520d-5p, mir-524-5p, mir-4724-3p, mir-1294, mir-143, mir-4770, mir-3659, mir-145, mir-3163, mir-181a, mir-181b, mir-181c, mir-181d, mir-4262, mir-4279, mir-144, mir-642b, mir-4742-3p, mir-3177-5p, mir-656, mir-3121-3p, mir-106a, mir-106b, mir-17, mir-20a, mir-20b, mir-519d, mir-93, mir-1297, mir-26a, mir-26b, mir-4465, mir-326, mir-330-5p, mir-3927 and mir-2113.

Additional miRNAs contemplated for upregulation include, mir-372, mir-373, mir-520a-3p, mir-520b, mir-520c-3p, mir-520d-3p, mir-520e, mir-199a-3p, mir-199b-3p, mir-3129-5p.

The upregulation may be effected in vivo or ex vivo.

Mesenchymal stem cells give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the easily accessible fat tissue and BM far exceeds their abundance in other tissues and as such isolation from BM and fat tissue is presently preferred.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Mesenchymal stem cells may be isolated from various tissues including but not limited to bone marrow, peripheral blood, blood, placenta (e.g. chorionic and/or amniotic), cord blood, umbilical cord, amniotic fluid and from adipose tissue.

A method of isolating mesenchymal stem cells from peripheral blood is described by Kassis et al [Bone Marrow Transplant. 2006 May; 37(10):967-76]. A method of isolating mesenchymal stem cells from placental tissue is described by Zhang et al [Chinese Medical Journal, 2004, 117 (6):882-887]. Methods of isolating and culturing adipose tissue, placental and cord blood mesenchymal stem cells are described by Kern et al [Stem Cells, 2006; 24:1294-1301].

According to a preferred embodiment of this aspect of the present invention, the mesenchymal stem cells are human.

According to another embodiment of this aspect of the present invention, the mesenchymal stem cells are isolated from placenta and umbilical cord of newborn humans.

Bone marrow can be isolated from the iliac crest of an individual by aspiration. Low-density BM mononuclear cells (BMMNC) may be separated by a FICOL-PAQUE density gradient or by elimination of red blood cells using Hetastarch (hydroxyethyl starch). Preferably, mesenchymal stem cell cultures are generated by diluting BM aspirates (usually 20 ml) with equal volumes of Hank's balanced salt solution (HBSS; GIBCO Laboratories, Grand Island, N.Y., USA) and layering the diluted cells over about 10 ml of a Ficoll column (Ficoll-Paque; Pharmacia, Piscataway, N.J., USA). Following 30 minutes of centrifugation at 2,500×g, the mononuclear cell layer is removed from the interface and suspended in HBSS. Cells are then centrifuged at 1,500×g for 15 minutes and resuspended in a complete medium (MEM, a medium without deoxyribonucleotides or ribonucleotides; GIBCO); 20% fetal calf serum (FCS) derived from a lot selected for rapid growth of MSCs (Atlanta Biologicals, Norcross, Ga.); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, N.Y.) and incubated at 37° C. with 5% humidified CO2. Following 24 hours in culture, non-adherent cells are discarded, and the adherent cells are thoroughly washed twice with phosphate buffered saline (PBS). The medium is replaced with a fresh complete medium every 3 or 4 days for about 14 days.

Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, Pa.). Cultured cells are recovered by centrifugation and resuspended with 5% DMSO and 30% FCS at a concentration of 1 to 2×106 cells per ml. Aliquots of about 1 ml each are slowly frozen and stored in liquid nitrogen.

Adipose tissue-derived MSCs can be obtained by liposuction and mononuclear cells can be isolated manually by removal of the fat and fat cells or using the Celution System (Cytori Therapeutics) following the same procedure as described above for preparation of MSCs.

According to one embodiment the populations are plated on polystyrene plastic surfaces (e.g. in a flask) and mesenchymal stem cells are isolated by removing non-adherent cells. Alternatively, mesenchymal stem cell may be isolated by FACS using mesenchymal stem cell markers.

Preferably the MSCs are at least 50% purified, more preferably at least 75% purified and even more preferably at least 90% purified.

To expand the mesenchymal stem cell fraction, frozen cells are thawed at 37° C., diluted with a complete medium and recovered by centrifugation to remove the DMSO.

Cells are resuspended in a complete medium and plated at a concentration of about 5,000 cells/cm2. Following 24 hours in culture, non-adherent cells are removed, and the adherent cells are harvested using Trypsin/EDTA, dissociated by passage through a narrowed Pasteur pipette, and preferably replated at a density of about 1.5 to about 3.0 cells/cm2. Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000].

MSC cultures utilized by some embodiments of the invention preferably include three groups of cells which are defined by their morphological features: small and agranular cells (referred to as RS-1, herein below), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

When MSCs are cultured under the culturing conditions of some embodiments of the invention they exhibit negative staining for the hematopoietic stem cell markers CD34, CD11B, CD43 and CD45. A small fraction of cells (less than 10%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Stro1 markers and are dimly positive for the CD90 marker, and the RS-2 cells are negative for all of these markers.

The mesenchymal stem cells of the present invention may be of autologous, syngeneic or allogeneic related (matched siblings or haploidentical family members) or unrelated fully mismatched source, as further described herein below.

Culturing of the mesenchymal stem cells can be performed in any media that support (or at least does not inhibit) the differentiation of the cells towards astrocytic cells such as those described in U.S. Pat. No. 6,528,245 and by Sanchez-Ramos et al. (2000); Woodburry et al. (2000); Woodburry et al. (J. Neurosci. Res. 96:908-917, 2001); Black and Woodbury (Blood Cells Mol. Dis. 27:632-635, 2001); Deng et al. (2001), Kohyama et al. (2001), Reyes and Verfatile (Ann N.Y. Acad. Sci. 938:231-235, 2001) and Jiang et al. (Nature 418:47-49, 2002).

The media may be G5, neurobasal medium, DMEM or DMEM/F12, OptiMEM™ or any other medium that supports neuronal or astrocytic growth.

According to a particular embodiment the miRNA comprises at least one of miR-20b, miR-146, miR-101 and miR-141.

A particular combination contemplated by the present inventors includes up-regulating each of miR-20b, miR-101 and miR-146a in the MSC population.

Another combination contemplated by the present inventors is up-regulating the level of exogenous miR-9 and exogenous miR-20b in the MSC population.

The term “microRNA”, “miRNA”, and “miR” are synonymous and refer to a collection of non-coding single-stranded RNA molecules of about 19-28 nucleotides in length, which regulate gene expression. MiRNAs are found in a wide range of organisms and have been shown to play a role in development, homeostasis, and disease etiology.

Below is a brief description of the mechanism of miRNA activity.

Genes coding for miRNAs are transcribed leading to production of a miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nt precursor known as the pre-miRNA. Drosha cleaves the pri-miRNA with a staggered cut typical of RNase III endonucleases yielding a pre-miRNA stem loop with a 5′ phosphate and ^{˜}2 nucleotide 3′ overhang. It is estimated that approximately one helical turn of stem (^{˜}10 nucleotides) extending beyond the Drosha cleavage site is essential for efficient processing. The pre-miRNA is then actively transported from the nucleus to the cytoplasm by Ran-GTP and the export receptor exportin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ^{˜}2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC).

Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA: miRNA* duplex is loaded into the RISC, the miRNA* is removed and degraded. The strand of the miRNA: miRNA* duplex that is loaded into the RISC is the strand whose 5′ end is less tightly paired. In cases where both ends of the miRNA: miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 Genes Dev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O, 4′-C-ethylene-bridged nucleic acids (ENA)). Other modifications are described herein below. For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise any of the sequences of the disclosed miRNAs, or variants thereof.

It will be appreciated from the description provided herein above, that contacting mesenchymal stem cells may be affected in a number of ways:

1. Transiently transfecting the mesenchymal stem cells with the mature miRNA (or modified form thereof, as described herein below). The miRNAs designed according to the teachings of the present invention can be generated according to any oligonucleotide synthesis method known in the art, including both enzymatic syntheses and solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

2. Stably, or transiently transfecting the mesenchymal stem cells with an expression vector which encodes the mature miRNA.

3. Stably, or transiently transfecting the mesenchymal stem cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of the miRNA.

4. Stably, or transiently transfecting the mesenchymal stem cells with an expression vector which encodes the pri-miRNA. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. Preparation of miRNAs mimics can be affected by chemical synthesis methods or by recombinant methods.

As mentioned, the present invention also contemplates differentiation of mesenchymal stem cells towards an astrocytic phenotype by down-regulation of particular miRNAs-namely mi-R-193b, mi-R-221, mi-R-135a, mi-R-149, mi-R-222, mi-R-199a, mi-R-302, mi-R-302c, mi-R-302d, mi-R-369-3p, mi-R-370, mi-R-let7a, mi-R-let7b, mi-R-10b, mi-R-23a, mi-R-23b, mi-R-32, miR-145, miR-133, mi-R-138, mi-R-182, mi-R-487, mi-R-214, mi-R-409, mi-R-548-d1, mi-R-889, as well as mi-R-1238.

Additional miRNAs contemplated for down-regulation are set forth below. miR-204, miR-224, miR-616, miR-122, miR-299, miR-100, miR-138, miR-140, miR-375, miR-217, miR-302, miR-372, miR-96, miR-127-3p, miR-449, miR-135b, miR-101, miR-326, miR-324, miR-335, miR-14, miR-16.

Additional miRNAs contemplated for down-regulation are set forth below. mir-410, mir-3163, mir-148a, mir-148b, mir-152, mir-3121-3p, mir-495, mir-203, mir-4680-3p.

According to a particular embodiment, at least one of miR-32, miR-221, miR-302a, miR-138 and miR-302b is down-regulated in order to produce the astrocyte-like cells of the present invention.

Down-regulating miRNAs can be affected using a polynucleotide which is hybridizable in cells under physiological conditions to the miRNA.

According to a particular embodiment, the cell population is generated by up-regulating an expression of miR-9, miR-146 and miR-101 in a population of MSCs and down-regulating an expression of miR-10b and miR-302 in the population of MSCs.

According to another embodiment, the cell population is generated by up-regulating an expression of miR-101 and down-regulating an expression of miR-138.

As used herein, the term “hybridizable” refers to capable of hybridizing, i.e., forming a double strand molecule such as RNA:RNA, RNA:DNA and/or DNA:DNA molecules. “Physiological conditions” refer to the conditions present in cells, tissue or a whole organism or body. Preferably, the physiological conditions used by the present invention include a temperature between 34-40° C., more preferably, a temperature between 35-38° C., more preferably, a temperature between 36 and 37.5° C., most preferably, a temperature between 37 to 37.5° C.; salt concentrations (e.g., sodium chloride NaCl) between 0.8-1%, more preferably, about 0.9%; and/or pH values in the range of 6.5-8, more preferably, 6.5-7.5, most preferably, pH of 7-7.5.

As mentioned herein above, the polynucleotides which downregulate the above list of miRNAs and the miRNAs described herein above may be provided as modified polynucleotides using various methods known in the art.

For example, the oligonucleotides (e.g. miRNAs) or polynucleotides of the present invention may comprise heterocyclic nucleosides consisting of purines and the pyrimidines bases, bonded in a 3′-to-5′ phosphodiester linkage.

Preferably used oligonucleotides or polynucleotides are those modified either in backbone, internucleoside linkages, or bases, as is broadly described herein under.

Specific examples of preferred oligonucleotides or polynucleotides useful according to this aspect of the present invention include oligonucleotides or polynucleotides containing modified backbones or non-natural internucleoside linkages.

Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide or polynucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide or polynucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides or polynucleotides which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

Oligonucleotides or polynucleotides of the present invention may also include base modifications or substitutions. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and 0-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications.

To express miRNAs or polynucleotide agents which regulate miRNAs in mesenchymal stem cells, a polynucleotide sequence encoding the miRNA (or pre-miRNA, or pri-miRNA, or polynucleotide which down-regulates the miRNAs) is preferably ligated into a nucleic acid construct suitable for mesenchymal stem cell expression. Such a nucleic acid construct includes a promoter sequence for directing transcription of the polynucleotide sequence in the cell in a constitutive or inducible manner.

It will be appreciated that the nucleic acid construct of some embodiments of the invention can also utilize miRNA homologues which exhibit the desired activity (i.e., astrocytic differentiating ability). Such homologues can be, for example, at least 80%, at least 81%, at least 82%, at least 83%, at least 84%, at least 85%, at least 86%, at least 8′7%, at least 88%, at least 89%, at least 90%, at least 91%, at least 92%, at least 93%, at least 94%, at least 95%, at least 96%, at least 9′7%, at least 98%, at least 99% or 100% identical to any of the sequences provided, as determined using the BestFit software of the Wisconsin sequence analysis package, utilizing the Smith and Waterman algorithm, where gap weight equals 50, length weight equals 3, average match equals 10 and average mismatch equals −9.

In addition, the homologues can be, for example, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any of the sequences provided herein, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with some embodiments of the invention are promoter sequences which are active under most environmental conditions and most types of cells such as the cytomegalovirus (CMV) and Rous sarcoma virus (RSV).

Inducible promoters suitable for use with some embodiments of the invention include for example tetracycline-inducible promoter (Zabala M, et al., Cancer Res. 2004, 64(8): 2799-804).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed—i.e. mesenchymal stem cells.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

According to one embodiment, a lentiviral vector is used to transfect the mesenchymal stem cells.

Various methods can be used to introduce the expression vector of some embodiments of the invention into mesenchymal stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Bliotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Other vectors can be used that are non-viral, such as cationic lipids, polylysine, and dendrimers.

The miRNAs, miRNA mimics and pre-miRs can be transfected into cells also using nanoparticles such as gold nanoparticles and by ferric oxide magnetic NP˜see for example Ghosh et al., Biomaterials. 2013 January; 34(3):807-16; Crew E, et al., Anal Chem. 2012 Jan. 3; 84(1):26-9. As mentioned herein above, the polynucleotides which down-regulate the miRNAs described herein above may be provided as modified polynucleotides using various methods known in the art.

Other modes of transfection that do not involved integration include the use of minicircle DNA vectors or the use of PiggyBac transposon that allows the transfection of genes that can be later removed from the genome.

As mentioned hereinabove, a variety of prokaryotic or eukaryotic cells can be used as host-expression systems to express the miRNAs or polynucleotide agent capable of down-regulating the miRNA of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the miRNAs of some embodiments of the invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

The conditions used for contacting the mesenchymal stem cells are selected for a time period/concentration of cells/concentration of miRNA/ratio between cells and miRNA which enable the miRNA (or inhibitors thereof) to induce differentiation thereof. The present invention further contemplates incubation of the mesenchymal stem cells with a differentiation factor which promotes differentiation towards an astrocytic lineage. The incubation with such differentiation factors may be affected prior to, concomitant with or following the contacting with the miRNA. According to this embodiment the medium may be supplemented with at least one of SHH (e.g. about 250 ng/ml), FGFb (e.g. 50 ng/ml), EGF (e.g. about 50 ng/ml), a cAMP inducer (e.g. IBMX or dbcycAMP), PDGF (e.g. about 5 ng/ml) neuregulin (e.g. about 50 ng/ml) and FGFb (e.g. about 20 ng/ml).

Alternatively, or additionally, the mesenchymal stem cells may be genetically modified so as to express such differentiation factors, using expression constructs such as those described herein above.

During or following the differentiation step the mesenchymal stem cells may be monitored for their differentiation state. Cell differentiation can be determined upon examination of cell or tissue-specific markers which are known to be indicative of differentiation. For example, the differentiated cells may express the following markers: S100 beta, glial fibrillary acidic protein (GFAP), glutamine synthetase, GLT-1, Excitatory Amino Acid Transporter 1 (EAAT1) and Excitatory Amino Acid Transporter 2 (EAAT2). Further, the differentiated cells may secrete a neurotrophic factor including for example glial derived neurotrophic factor (GDNF), GenBank accession nos. L19063, L15306; nerve growth factor (NGF), GenBank accession no. CAA37703; brain-derived neurotrophic factor (BDNF), GenBank accession no CAA62632; neurotrophin-3 (NT-3), GenBank Accession No. M37763; neurotrophin-4/5; Neurturin (NTN), GenBank Accession No. NP-004549; Neurotrophin-4, GenBank Accession No. M86528; Persephin, GenBank accession no. AAC39640; brain derived neurotrophic factor, (BDNF), GenBank accession no. CAA42761; artemin (ART), GenBank accession no. AAD13110; ciliary neurotrophic factor (CNTF), GenBank accession no. NP-000605; insulin growth factor-I (IGF-1), GenBank accession no. NP-000609; and/or Neublastin GenBank accession no. AAD21075.

It will be appreciated that the differentiation time may be selected so as to obtain early progenitors of astrocytes or more mature astrocytes. Enrichment for a particular early or mature astrocytic cell is also contemplated. Selection for cells which express markers such as CD44, A2B5 and S100 allows for the enrichment of progenitor type astrocytes, whereas selection for cells which express markers such as GFAP and glutamine synthase allows for selection of mature astrocytes.

Tissue/cell specific markers can be detected using immunological techniques well known in the art [Thomson J A et al., (1998). Science 282: 1145-7]. Examples include, but are not limited to, flow cytometry for membrane-bound markers, immunohistochemistry for extracellular and intracellular markers and enzymatic immunoassay, for secreted molecular markers.

In addition, cell differentiation can be also followed by specific reporters that are tagged with GFP or RFP and exhibit increased fluorescence upon differentiation.

Isolated cell populations obtained according to the methods describe herein are typically non-homogeneous, although homogeneous cell populations are also contemplated.

According to a particular embodiment, the cell populations are genetically modified to express a miRNA or a polynucleotide agent capable of down-regulating the miRNA.

The term “isolated” as used herein refers to a population of cells that has been removed from its in-vivo location (e.g. bone marrow, neural tissue). Preferably the isolated cell population is substantially free from other substances (e.g., other cells) that are present in its in-vivo location.

Cell populations may be selected such that more than about 50% of the cells express at least one, at least two, at least three, at least four, at least five or all of the following markers: S100 beta, glial fibrillary acidic protein (GFAP), glutamine sythetase, GLT-1, GDNF, BDNF, IGF-1 and GLAST.

Cell populations may be selected such that more than about 60% of the cells express at least one, at least two, at least three, at least four, at least five or all of the following markers: S100 beta, glial fibrillary acidic protein (GFAP), glutamine sythetase, GLT-1, GDNF, BDNF, IGF-1 and GLAST.

Cell populations may be selected such that more than about 70% of the cells express at least one, at least two, at least three, at least four, at least five or all of the following markers: S100 beta, glial fibrillary acidic protein (GFAP), glutamine sythetase, GLT-1, GDNF, BDNF, IGF-1 and GLAST.

Cell populations may be selected such that more than about 80% of the cells express at least one, at least two, at least three, at least four, at least five or all of the following markers: S100 beta, glial fibrillary acidic protein (GFAP), glutamine sythetase, GLT-1, GDNF, BDNF, IGF-1 and GLAST.

Cell populations may be selected such that more than about 90% of the cells express at least one, at least two, at least three, at least four, at least five or all of the following markers: S100 beta, glial fibrillary acidic protein (GFAP), glutamine sythetase, GLT-1, GDNF, BDNF, IGF-1 and GLAST.

Cell populations may be selected such that more than about 95% of the cells express at least one, at least two, at least three, at least four, at least five or all of the following markers: S100 beta, glial fibrillary acidic protein (GFAP), glutamine sythetase, GLT-1, GDNF, BDNF, IGF-1 and GLAST.

Isolation of particular subpopulations of cells may be effected using techniques known in the art including fluorescent activated cell sorting and/or magnetic separation of cells.

The cells of the populations of this aspect of the present invention may comprise structural astrocytic phenotypes including a cell size, a cell shape, an organelle size and an organelle number. Thus, mature astrocytic structural phenotypes include a round nucleus, a “star shaped” body and many long processes that end as vascular foot plates on the small blood vessels of the CNS.

These structural phenotypes may be analyzed using microscopic techniques (e.g. scanning electron microscopy). Antibodies or dyes may be used to highlight distinguishing features in order to aid in the analysis.

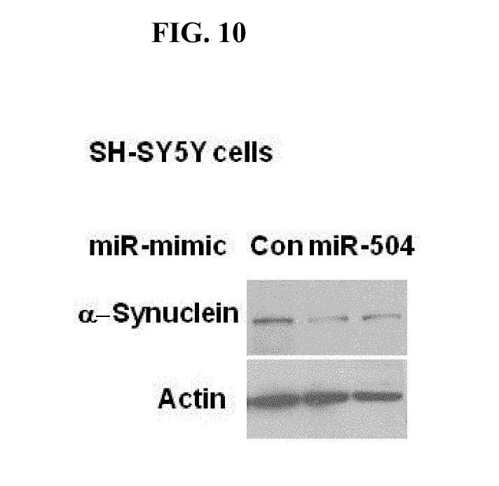

The present inventors have further shown that a particular miRNA (miRNA 504) which is upregulated on differentiation of MSCs towards an astrocytic phenotype targets α-Synuclein (see FIG. 10). Mutations within the α-Synuclein gene are associated with autosomal dominant familial PD.

Thus, the present inventors further propose use of MSCs as a cargo cell to transport miRNA 504 to the brain where the miRNA then targets the α-Synuclein as a treatment for Parkinson's.

Another miRNA (miRNA 152) which is upregulated on differentiation of MSCs towards an astrocytic phenotype targets Huntingdon (HTT) gene. Mutations within this gene are associated with Huntingdon disease (HD).

Thus, the present inventors further propose use of MSCs as a cargo cell to transport miRNA 152 to the brain where the miRNA then targets the α-Synuclein as a treatment for Huntingdon's disease.

Another miRNA (miRNA 665) which is upregulated on differentiation of MSCs towards an astrocytic phenotype targets the prion gene (PRNP). Thus, the present inventors further propose use of MSCs as a cargo cell to transport miRNA 665 to the brain where the miRNA then targets PRNP.

Another miRNA (miRNA 340) which is upregulated on differentiation of MSCs towards an astrocytic phenotype targets SOD1 gene. Mutations within this gene are associated with ALS.

Thus, the present inventors further propose use of MSCs as a cargo cell to transport miRNA 340 to the brain where the miRNA then targets the SOD1 gene as a treatment for ALS.

According to this aspect of the invention, the MSCs may be manipulated to express the miRNA (or mimic thereof) and cultured so that they differentiate towards the astrocytic phenotype as described herein above. Alternatively, the MSCs may be manipulated to express the miRNA (or mimic thereof) and administered to the patient (e.g. a patient with Parkinson's) without allowing for astrocytic differentiation.

The cells and cell populations of the present invention may be useful for a variety of therapeutic purposes. Representative examples of CNS diseases or disorders that can be beneficially treated with the cells described herein include, but are not limited to, a pain disorder, a motion disorder, a dissociative disorder, a mood disorder, an affective disorder, a neurodegenerative disease or disorder and a convulsive disorder.

More specific examples of such conditions include, but are not limited to, Parkinson's, ALS, Multiple Sclerosis, Huntingdon's disease, autoimmune encephalomyelitis, diabetic neuropathy, glaucatomus neuropathy, macular degeneration, action tremors and tardive dyskinesia, panic, anxiety, depression, alcoholism, insomnia, manic behavior, Alzheimer's and epilepsy.

The use of differentiated MSCs may be also indicated for treatment of traumatic lesions of the nervous system including spinal cord injury and also for treatment of stroke caused by bleeding or thrombosis or embolism because of the need to induce neurogenesis and provide survival factors to minimize insult to damaged neurons.

In any of the methods described herein the cells may be obtained from an autologous, semi-autologous or non-autologous (i.e., allogeneic or xenogeneic) human donor or embryo or cord/placenta. For example, cells may be isolated from a human cadaver or a donor subject.

The term semi-autologous refers to donor cells which are partially-mismatched to recipient cells at a major histocompatibility complex (MHC) class I or class II locus.

The cells of the present invention can be administered to the treated individual using a variety of transplantation approaches, the nature of which depends on the site of implantation.

The term or phrase “transplantation”, “cell replacement” or “grafting” are used interchangeably herein and refer to the introduction of the cells of the present invention to target tissue. As mentioned, the cells can be derived from the recipient or from an allogeneic, semi-allogeneic or xenogeneic donor.

The cells can be injected systemically into the circulation, administered intrathecally or grafted into the central nervous system, the spinal cord or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the brain parenchyma at the time of transplantation.

Intraparenchymal transplantation can be performed using two approaches: (i) injection of cells into the host brain parenchyma or (ii) preparing a cavity by surgical means to expose the host brain parenchyma and then depositing the graft into the cavity.

Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura.

Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The cells may also be transplanted to a healthy region of the tissue. In some cases, the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions.

Multiple grafts may consist of a mixture of cell types, and/or a mixture of transgenes inserted into the cells. Preferably from approximately 104 to approximately 109 cells are introduced per graft. Cells can be administered concomitantly to different locations such as combined administration intrathecally and intravenously to maximize the chance of targeting into affected areas.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20, 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated. Demyelinated MS lesions are distributed across multiple locations throughout the CNS, such that effective treatment of MS may rely more on the migratory ability of the cells to the appropriate target sites.

Intranasal administration of the cells described herein is also contemplated.

MSCs typically down regulate MHC class 2 and are therefore less immunogenic. Embryonal or newborn cells obtained from the cord blood, cord's Warton's gelly or placenta are further less likely to be strongly immunogenic and therefore less likely to be rejected, especially since such cells are immunosuppressive and immunoregulatory to start with.

Notwithstanding, since non-autologous cells may induce an immune reaction when administered to the body several approaches have been developed to reduce the likelihood of rejection of non-autologous cells. Furthermore, since diseases such as multiple sclerosis are inflammatory based diseases, the problem of immune reaction is exacerbated. These include either administration of cells to privileged sites, or alternatively, suppressing the recipient's immune system, providing anti-inflammatory treatment which may be indicated to control autoimmune disorders to start with and/or encapsulating the non-autologous/semi-autologous cells in immunoisolating, semipermeable membranes before transplantation.

As mentioned herein above, the present inventors also propose use of newborn mesenchymal stem cells to limit the immune reaction.

The following experiments may be performed to confirm the potential use of newborn's MSCs isolated from the cord/placenta for treatment of neurological disorders:

1) Differentiated MSCs (to various neural cells or neural progenitor cells) may serve as stimulators in one way mixed lymphocyte culture with allogeneic T cells and proliferative responses in comparison with T cells responding against allogeneic lymphocytes isolated from the same donor may be evaluated by 3H-Thymidine uptake to document hyporesponsiveness.

2) Differentiated MSCs may be added/co-cultured to one way mixed lymphocyte cultures and to cell cultures with T cell mitogens (phytohemmaglutinin and concanavalin A) to confirm the immunosuppressive effects on proliferative responses mediated by T cells.

3) Cord and placenta cells cultured from Brown Norway rats (unmodified and differentiated), may be enriched for MSCs and these cells may be infused into Lewis rats with induced experimental autoimmune encephalomyelitis (EAE). Alternatively, cord and placenta cells cultured from BALB/c mice, (BALB/cxC57BL/6)F1 or xenogeneic cells from Brown Norway rats (unmodified and differentiated), may be enriched for MSCs and these cells may be infused into C57BL/6 or SJL/j recipients with induced experimental autoimmune encephalomyelitis (EAE). The clinical effects against paralysis may be investigated to evaluate the therapeutic effects of xenogeneic, fully MHC mismatched or haploidentically mismatched MSCs. Such experiments may provide the basis for treatment of patients with a genetic disorder or genetically proned disorder with family member's haploidentical MSCs.

4) BALB/c MSCs cultured from cord and placenta may be transfused with pre-miR labeled with GFP or RFP, which will allow the inventors to follow the migration and persistence of these cells in the brain of C57BL/6 recipients with induced EAE. The clinical effects of labeled MHC mismatched differentiated MSCs may be evaluated by monitoring signs of disease, paralysis and histopathology. The migration and localization of such cells may be also monitored by using fluorescent cells from genetically transduced GFP “green” or Red2 “red” donors.

As mentioned, the present invention also contemplates encapsulation techniques to minimize an immune response.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J. Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 .mu.m.

Such microcapsules can be further encapsulated with additional 2-5 .mu.m ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 .mu.m (Canaple L. et al, Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE™), etanercept, TNF alpha blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

In any of the methods described herein, the cells can be administered either per se or, preferably as a part of a pharmaceutical composition that further comprises a pharmaceutically acceptable carrier.

As used herein a “pharmaceutical composition” refers to a preparation of one or more of the cell compositions described herein, with other chemical components such as pharmaceutically suitable carriers and excipients. The purpose of a pharmaceutical composition is to facilitate administration of the cells to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, saline, emulsions and mixtures of organic solvents with water.

Herein the term “excipient” refers to an inert substance added to a pharmaceutical composition to further facilitate administration of a compound.

Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration include direct administration into the circulation (intravenously or intra-arterial), into the spinal fluid or into the tissue or organ of interest. Thus, for example the cells may be administered directly into the brain.

For any preparation used in the methods of the invention, the therapeutically effective amount or dose can be estimated initially from in vitro and cell culture assays.

Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, animal models of demyelinating diseases include shiverer (shi/shi, MBP deleted) mouse, MD rats (PLP deficiency), Jimpy mouse (PLP mutation), dog shaking pup (PLP mutation), twitcher mouse (galactosylceramidase defect, as in human Krabbe disease), trembler mouse (PMP-22 deficiency). Virus induced demyelination model comprise use if Theiler's virus and mouse hepatitis virus.

Autoimmune EAE is a possible model for multiple sclerosis.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, a multiple sclerosis patient can be monitored symptomatically for improved motor functions indicating positive response to treatment.

For injection, the active ingredients of the pharmaceutical composition may be formulated in aqueous solutions, preferably in physiologically compatible buffers such as Hank's solution, Ringer's solution, or physiological salt buffer.

Dosage amount and interval may be adjusted individually to levels of the active ingredient which are sufficient to effectively treat the brain disease/disorder. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated multiple sclerosis patient will be administered with an amount of cells which is sufficient to alleviate the symptoms of the disease, based on the monitoring indications.

The cells of the present invention may be co-administered with therapeutic agents useful in treating neurodegenerative disorders, such as gangliosides; antibiotics, neurotransmitters, neurohormones, toxins, neurite promoting molecules; and antimetabolites and precursors of neurotransmitter molecules such as L-DOPA.

As used herein the term “about” refers to +/−10%.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In some embodiments, a neurodegenerative disease or condition comprises alpha-synucleinopathies. Non-limiting examples of alpha-synucleinopathies include, but are not limited to Parkinson's disease, multiple system atrophy, and Dementia with Lewy bodies.

In some embodiments, the disease is a disease characterized or caused by alpha-synuclein or elevated levels of alpha-synuclein. In some embodiments, the disease characterized by alpha-synuclein is Parkinson's disease. In some embodiments, the disease is characterized or caused by the presence of Lewy bodies. In some embodiments, the disease is selected from Parkinson's disease, multiple system atrophy and dementia with Lewy bodies. In some embodiments, the disease is selected from multiple system atrophy and dementia with Lewy bodies.

In some embodiments, a neurodegenerative disease or condition comprises any disease or condition comprising the appearance of A1 reactive astrocytes. Methods for identifying A1 astrocytes would be apparent to one of ordinary skill in the art, and can be utilized to detect A1 specific markers, including but are not limited to C3, C4B and CXCL10.

In some embodiments, the present invention is directed to an isolated population of MSCs, and/or exosome derived therefrom, comprising an exogenous miR-504. In some embodiments, the MSC further comprises an RNA oligonucleotide that hybridizes to an inhibits miR-302. In some embodiments, the RNA oligonucleotide is an antagomir. In some embodiments, an MSC population is differentiated toward an astrocyte phenotype. In some embodiments, the isolated population is of genetically modified MSCs differentiated toward an astrocyte phenotype.

In some embodiments, miR-504 is hsa-miR-504-3p. In some embodiments, miR-504 comprises hsa-miR-504-3p. In some embodiments, miR-504 is hsa-miR-504-5p. In some embodiments, miR-504 comprises hsa-miR-504-5p. In some embodiments, hsa-miR-504-3p is denoted by MIMAT0026612. In some embodiments, the sequence of hsa-miR-504-3p is GGGAGUGCAGGGCAGGGUUUC (SEQ ID NO: 481). In some embodiments, hsa-miR-504-5p is denoted by MIMAT0002875. In some embodiments, the sequence of hsa-miR-504-5p is AGACCCUGGUCUGCACUCUAUC (SEQ ID NO: 482). In some embodiments, the pre-miR of miR-504 is denoted by MI0003189. In some embodiments, the pre-miR of miR-504 comprises or consists of the sequence GCUGCUGUUGGGAGACCCUGGUCUGCACUCUAUCUGUAUUCUUACUGAAGG GAGUGCAGGGCAGGGUUUCCCAUACAGAGGGC (SEQ ID NO: 483).

In some embodiments, miR-302 is anyone of miR-302a, miR-302b, miR-302c, miR-302d, miR-302e, miR-302f. In some embodiments, miR-302 is miR-302a. In some embodiments, miR-302 is miR-302b. In some embodiments, miR-302 is miR-302c. In some embodiments, miR-302 is miR-302d. In some embodiments, miR-302 is miR-302e. In some embodiments, miR-302 is miR-302f. In some embodiments, miR-302a consists or comprises hsa-miR-302a-3p and/or hsa-miR-302a-5p. In some embodiments, miR-302b consists or comprises hsa-miR-302b-3p and/or hsa-miR-302b-5p. In some embodiments, miR-302c consists or comprises hsa-miR-302c-3p and/or hsa-miR-302c-5p. In some embodiments, miR-302d consists or comprises hsa-miR-302d-3p and/or hsa-miR-302d-5p.

In some embodiments, miR-302 is a miR-302 mimic comprising or consisting of the sequence UAAGUGCUUCCAUGUUUUGGUGA (SEQ ID NO: 484). In some embodiments, the antagomir and/or RNA oligonucleotide that binds to an inhibits miR-302 binds to and inhibits all forms of miR-302 described herein. In some embodiments, the antagomir and/or RNA oligonucleotide that binds to an inhibits miR-302 comprises or consists of the sequence AUUCACGAAGGUACAAAACCACU (SEQ ID NO: 485).

In some embodiments, hsa-miR-302a-3p is denoted by MIMAT0000684. In some embodiments, the sequence of hsa-miR-302a-3p is UAAGUGCUUCCAUGUUUUGGUGA (SEQ ID NO: 486). In some embodiments, hsa-miR-302a-5p is denoted by MIMAT0000683. In some embodiments, the sequence of hsa-miR-302a-5p is ACUUAAACGUGGAUGUACUUGCU (SEQ ID NO: 487). In some embodiments, the pre-miR of miR-302a is denoted by MI0000738. In some embodiments, the pre-miR of miR-302a comprises or consists of the sequence CCACCACUUAAACGUGGAUGUACUUGCUUUGAAACUAAAGAAGUAAGUGCU UCCAUGUUUUGGUGAUGG (SEQ ID NO: 488).

In some embodiments, hsa-miR-302b-3p is denoted by MIMAT0000715. In some embodiments, the sequence of hsa-miR-302b-3p is UAAGUGCUUCCAUGUUUUAGUAG (SEQ ID NO: 489). In some embodiments, hsa-miR-302b-5p is denoted by MIMAT0000714. In some embodiments, the sequence of hsa-miR-302b-5p is ACUUUAACAUGGAAGUGCUUUC (SEQ ID NO: 490). In some embodiments, the pre-miR of miR-302b is denoted by MI0000772. In some embodiments, the pre-miR of miR-302b comprises or consists of the sequence GCUCCCUUCAACUUUAACAUGGAAGUGCUUUCUGUGACUUUAAAAGUAAGU GCUUCCAUGUUUUAGUAGGAGU (SEQ ID NO: 491).

In some embodiments, hsa-miR-302c-3p is denoted by MIMAT0000717. In some embodiments, the sequence of hsa-miR-302c-3p is UAAGUGCUUCCAUGUUUCAGUGG (SEQ ID NO: 492). In some embodiments, hsa-miR-302c-5p is denoted by MIMAT0000716. In some embodiments, the sequence of hsa-miR-302c-5p is UUUAACAUGGGGGUACCUGCUG (SEQ ID NO: 493). In some embodiments, the pre-miR of miR-302c is denoted by MI0000773. In some embodiments, the pre-miR of miR-302c comprises or consists of the sequence CCUUUGCUUUAACAUGGGGGUACCUGCUGUGUGAAACAAAAGUAAGUGCUU CCAUGUUUCAGUGGAGG (SEQ ID NO: 494).

In some embodiments, hsa-miR-302d-3p is denoted by MIMAT0000718. In some embodiments, the sequence of hsa-miR-302d-3p is UAAGUGCUUCCAUGUUUGAGUGU (SEQ ID NO: 495). In some embodiments, hsa-miR-302d-5p is denoted by MIMAT0004685. In some embodiments, the sequence of hsa-miR-302d-5p is ACUUUAACAUGGAGGCACUUGC (SEQ ID NO: 496). In some embodiments, the pre-miR of miR-302d is denoted by MI0000774. In some embodiments, the pre-miR of miR-302d comprises or consists of the sequence CCUCUACUUUAACAUGGAGGCACUUGCUGUGACAUGACAAAAAUAAGUGCU UCCAUGUUUGAGUGUGG (SEQ ID NO: 497).

In some embodiments, hsa-miR-302e is denoted by MIMAT0005931. In some embodiments, the sequence of hsa-miR-302e is UAAGUGCUUCCAUGCUU (SEQ ID NO: 498). In some embodiments, the pre-miR of miR-302e is denoted by MI0006417. In some embodiments, the pre-miR of miR-302e comprises or consists of the sequence UUGGGUAAGUGCUUCCAUGCUUCAGUUUCCUUACUGGUAAGAUGGAUGUAG UAAUAGCACCUACCUUAUAGA (SEQ ID NO: 499).

In some embodiments, hsa-miR-302f is denoted by MIMAT0005932. In some embodiments, the sequence of hsa-miR-302f is UAAUUGCUUCCAUGUUU (SEQ ID NO: 500). In some embodiments, the pre-miR of miR-302f is denoted by MI0006418. In some embodiments, the pre-miR of miR-302f comprises or consists of the sequence UCUGUGUAAACCUGGCAAUUUUCACUUAAUUGCUUCCAUGUUUAUAAAAGA (SEQ ID NO: 501).

The term “extracellular vesicles”, as used herein, refers to all cell-derived vesicles secreted from MSCs including but not limited to exosomes and microvesicles. “Exosome”, as used herein, refers to cell-derived vesicles of endocytic origin, with a size of 50-100 nm, and secreted from MSCs. As a non-limiting embodiment, for the generation of exosomes cells are maintained with Opti-MEM and human serum albumin or 5% FBS that was depleted from exosomes. In some embodiments, exosomes comprise all extracellular vesicles.

Exosomes, and extracellular vesicles can be obtained by growing MSCs in culture medium with serum depleted from exosomes or in serum-free media such as OptiMeM and subsequently isolating the exosomes by ultracentrifugation. Other methods associated with beads, columns, filters and antibodies are also employed. In some embodiments, the cells are grown in hypoxic conditions or incubated in medium with low pH so as to increase the yield of the exosomes. In other embodiments, the cells are exposed to radiation so as to increases exosome secretion and yield. In some embodiments, the exosomes are suspended in appropriate carrier for administration.

In some embodiments, the astrocyte phenotype comprises expression of glial fibrillary acidic protein (GFAP). In some embodiments, at least 50% of the MSCs in the population express GFAP. In some embodiments, the MSC differentiated toward an astrocyte phenotype expresses excitatory amino acid transporter 2 (EAAT2) and/or glial cell-derived neurotrophic factor (GDNF). In some embodiments, at least 50% of the population expresses EAAT2 and/or GDNF. In some embodiments, the at least 50% of the population is identified by expression of a marker selected from protein S100, glutamine synthetase, EAAT1, EAAT2 and GDNF. In some embodiments, the at least 50% of the population is identified by expression of a marker selected from EAAT2, and GDNF.

In some embodiments, the method of generating the isolated population of the invention comprises introducing and expressing in MSCs an exogenous miR-504, thereby generating an isolated population of genetically modified MSCs differentiated toward an astrocyte phenotype. In some embodiments, the method further comprises introducing and/or expressing in the MSCs an antagomir to miR-302. In some embodiments, the method further comprises introducing and/or expressing in the MSCs an RNA oligonucleotide that hybridizes to an inhibits miR-302. In some embodiments, the introducing and expressing comprises transfecting the MSCs with an expression vector which comprises a polynucleotide sequence which encodes a pre-miRNA of the miR-504. In some embodiments, the introducing and expressing comprises transfecting the MSCs with an expression vector which comprises a polynucleotide sequence which encodes a polynucleotide sequence which encodes the miR-504. In some embodiments, the method further comprises analyzing expression of at least one marker selected from the group consisting of EAAT2 and GDNF. In some embodiments, the method further comprises analyzing expression of at least one marker selected from the group consisting of GDNF, S100, glutamine synthetase, EAAT1 and EAAT2. In some embodiments, the analyzing is following the generating. In some embodiments, the method further comprises incubating the MSCS in a differentiation medium.

In some embodiments, there is provided a pharmaceutical composition comprising an isolated population of the invention and a pharmaceutically acceptable carrier.

In some embodiments, there is provided a method of decreasing expression of alpha-synuclein (SNCA) in a target cell, the method comprising contacting the target cell with the isolated population or the pharmaceutical composition of the invention. In some embodiments, the isolated population or pharmaceutical composition comprises exogenous miR-504. In some embodiments, SNCA mRNA is decreased. In some embodiments, the SNCA protein is decreased. In some embodiments, the cell is in vitro. In some embodiments, the cell is in a subject. In some embodiments, the cell is a neuronal cell. In some embodiments, the cell comprises increased SNCA expression as compared to a healthy cell.

In some embodiments, there is provided a method of treating a SNCA-associated disease in a subject in need thereof, comprising administering to the subject a pharmaceutical composition of the invention. In some embodiment, the SNCA-associated disease is Parkinson's Disease. In some embodiments, the pharmaceutical composition comprises miR-504. In some embodiments, the pharmaceutical composition further comprises an antagomir and/or an RNA oligonucleotide that hybridizes to and inhibits miR-302. In some embodiments, the pharmaceutical composition comprises a therapeutically effective amount of MSCs. In some embodiments, the pharmaceutical composition comprises a therapeutically effective amount of MSCs, exosomes or a combination thereof. In some embodiments, the MSCs are autologous to the subject. In some embodiments, the MSCs are non-autologous to the subject. In some embodiments, the MSCs are semi-autologous to the subject. In some embodiments, the MSCs are autologous, non-autologous or semi-autologous to the subject.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is noted that for each miR described herein the corresponding sequence (mature and pre) is provided in the sequence listing which should be regarded as part of the specification.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells˜A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization˜A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1: Soluble Factors for the Differentiation of MSCs Towards an Astrocytic Phenotype

Materials and Methods

Differentiation of MSCs to Cells Expressing Astrocytic Phenotypes:

MSCs from the four different sources (bone marrow (BM-MSCs), adipose-derived (AD-MSCs), cord and placenta-derived cells) were employed in these studies. The cells were placed first in DMEM+10% FCS for 1 day and were then transferred for 5 days to NM media containing SHH 250 ng/ml, FGFb (50 ng/ml) and EGF 50 ng/ml. The cells were incubated for an additional 10 days with IBMX (0.5 mM), dbcycAMP (1 mM), PDGF (5 ng/ml) neuregulin (50 ng/ml) and FGFb (20 ng/ml). In the last stage, the cells were incubated for 5 days in G5 media supplemented with the same factors.

The differentiated cells were analyzed for the following markers:

Nestin, Olig2, β-III tubulin, GFAP, glutamine synthase.

Results

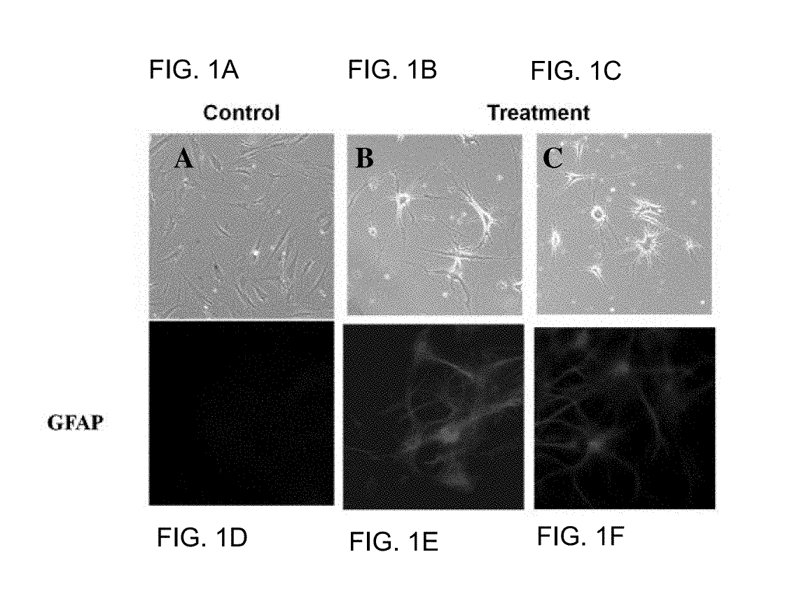

Using the above described differentiation protocols, both BM-MSC (FIG. 1) and the other MSC types (data not shown) exhibited astrocytic morphology and were stained positive for the astrocytic marker GFAP (FIG. 1).

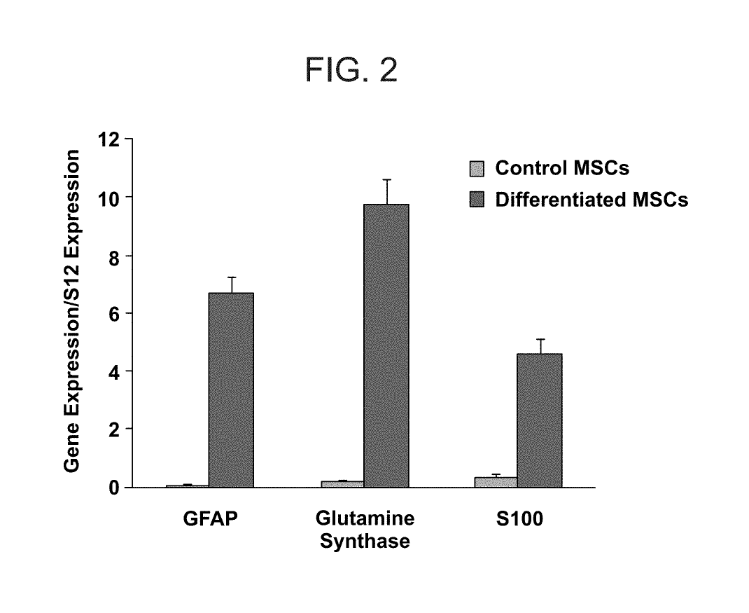

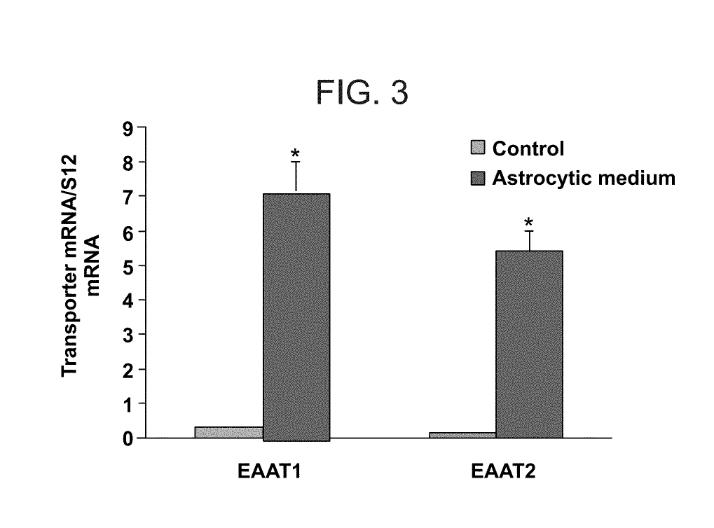

The present inventors further analyzed the differentiated cells and found that they expressed mRNA of GFAP and S100 as well as the glutamate transporters, as shown in FIGS. 2 and 3.

### Example 2: miRNAs for the Differentiation of MSCs into Astrocytes

Materials and Methods

miRNA Microarray Analysis:

For analyzing the differential expression of specific miRNA in control and differentiated MSCs, the Stem cell microRNA qPCR array was used, with quantiMiR from SBI company (catalog #RA620A-1).

The system allows for the ability to quantitate fold differences of 95 separate microRNAs between 2 separate experimental RNA samples. The array plate also includes the U6 transcript as a normalization signal. All 95 microRNAs chosen for the array have published implications with regard to potential roles in stem cell self-renewal, hematopoiesis, neuronal development and differentiated tissue identification.

Total RNA was isolated from 105-106 cells of control and differentiated MSCs using miRneasy total RNA isolation kit from Qiagen (catalog #217004) that isolate RNA fraction with sizes <200 bp.

500 ng of total RNA was processed according to “SBI Stem Cell MicroRNA qPCR Array with QuantiMir™” (Cat. # RA620A-1) user protocol, the contents of which are incorporated herein by reference. For the qPCR, the Applied Biosystems Power SYBR master mix (cat#4367659) was used.

For validation, sybr-green qPCR of the specific miRNA of interest was performed on the same RNA samples processed according to QIAGEN miScript System handbook (cat #218061 & 218073).

Hu hsa-miR MicroRNA Profiling Kit (System Biosciences) “SBI Stem Cell MicroRNA qPCR Array with QuantiMir™” (Cat. # RA620A-1) which detects the expression of 96 miRNAs, was used to profile the miRNAs in unmodified BM-MSC compared with MSCs differentiated to astrocytes. 500 ng of total RNA was tagged with poly(A) to its 3′ end by poly A polymerase, and reverse-transcribed with oligo-dT adaptors by QuantiMir RT technology. Expression levels of the miRNAs were measured by quantitative PCR using SYBR green reagent and VIIA7, Real-Time PCR System (Applied Biosystems). All miRNAs could be measured with miRNA specific forward primers and a universal reverse primer (SBI). Expression level of the miRNAs was normalized to U6 snRNA, using the comparative CT method for relative quantification as calculated with the following equation:

2−[(CT astrocyte diff miRNA−CT astrocyte endogenous control)−(CT DMEM miRNA−CT DMEM endogenous control)].

Results

To identify miRNAs that may be involved in the differentiation of MSCs into astrocytes, the miRNA signature of control unmodified MSCs was compared to MSCs differentiated into astrocytes.

A qRT-PCR microarray was analyzed that contained 96 miRNAs, all of which were related to stem cells and that were divided into subgroups based on their known association with stem cells, neural-related, hematopoietic and organ-related miRNAs.

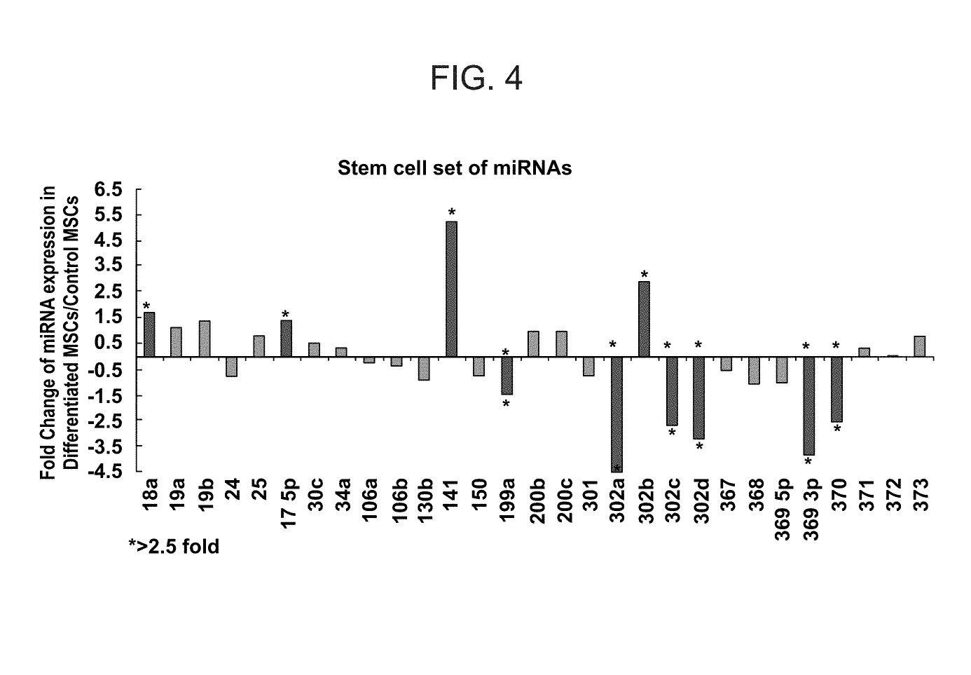

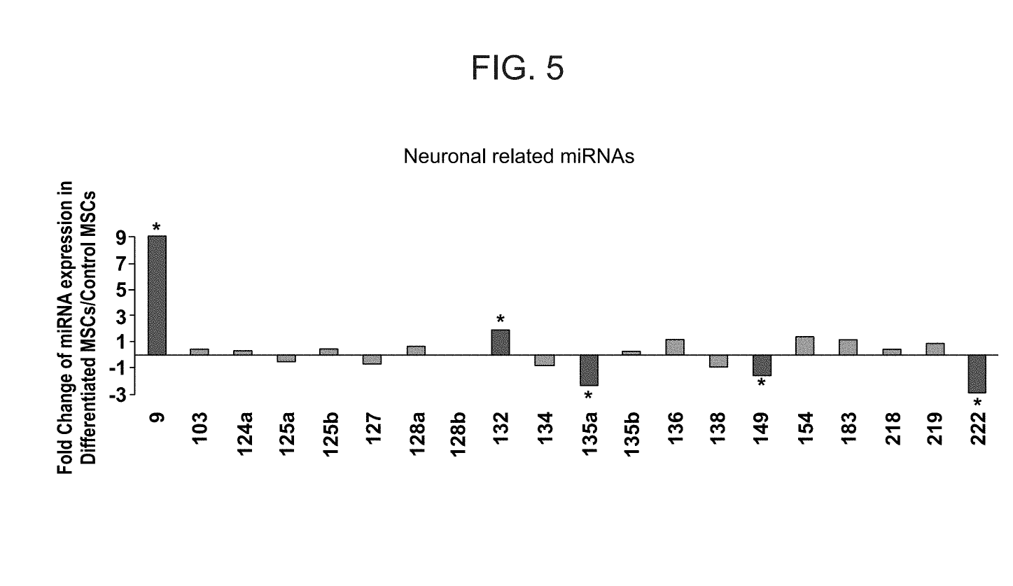

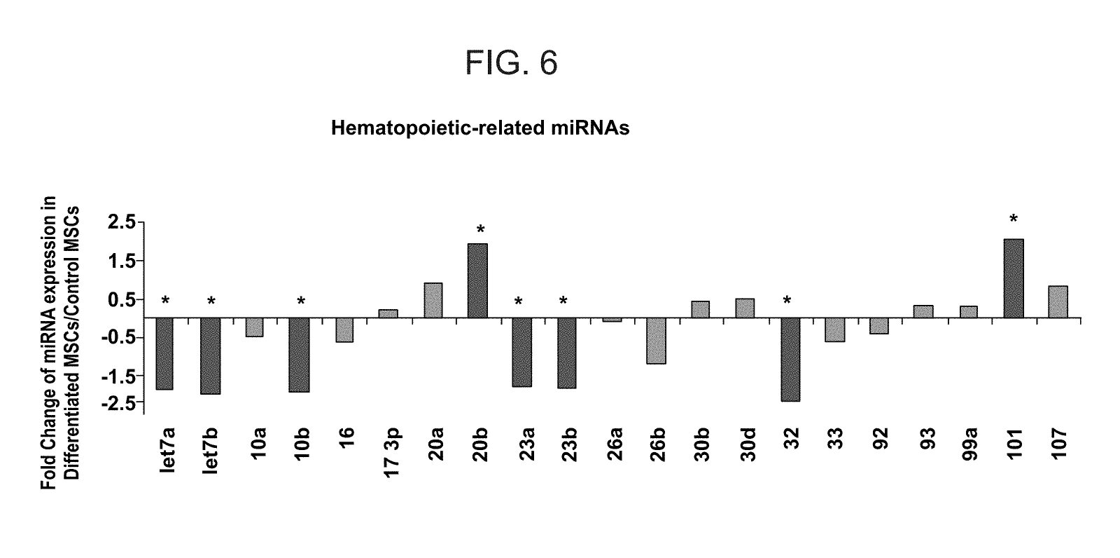

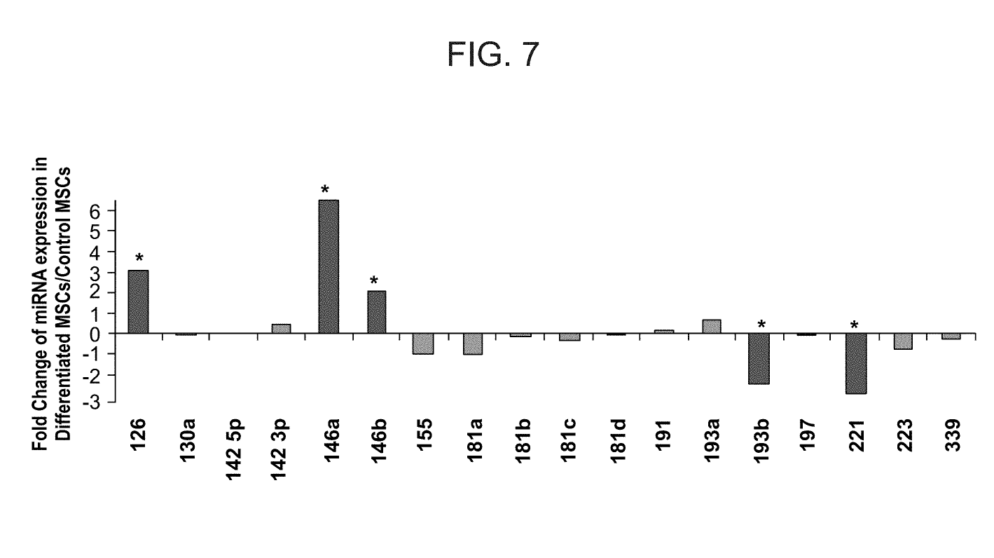

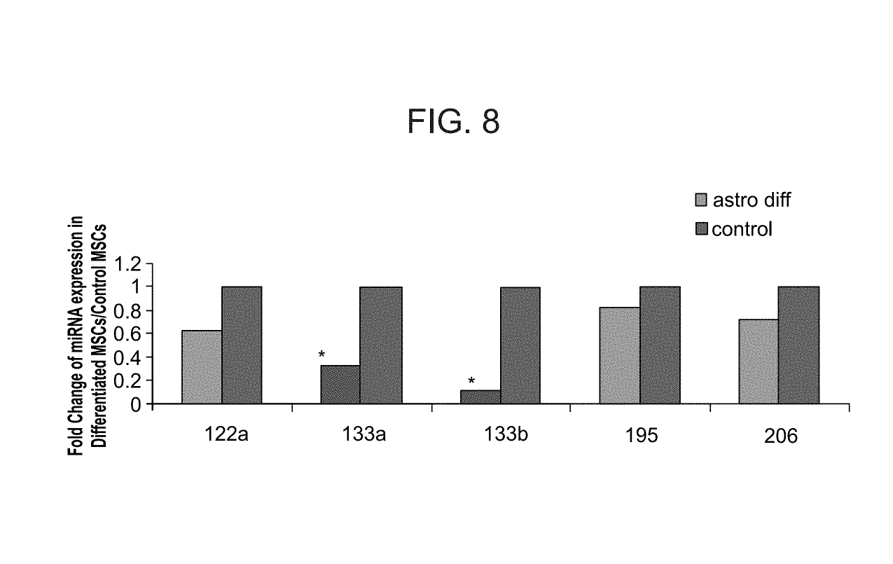

As presented in FIGS. 4-7, there were significant changes in the expression of specific miRNA of each group between the control MSCs and the differentiated ones.

qRT-PCR studies were then performed to validate the differences in the miRNA expression that were observed between the control and differentiated cells.

Similar to the results that were obtained with the microarray data, qRT-PCR it was found that the differentiated MSCs demonstrated a decrease in miRs, 32, 133, 221, 145, 302a and 302b and an increase in miRs 9, 20b, 101, 141, 146a and 146b.

The role of specific miRNAs in the astrocytic differentiation of the cells was further examined. It was found that the combination of miR-9 and miR-20b as well as combination of miR-20b, 101 and 146a also increased GFAP expression. Similarly, it was found that inhibiting miR-10b and miR-302 and expressing miR-9, 146 and 101 also increased GFAP expression (data not shown).

### Example 3 Identification of Additional miRNAs for the Differentiation of MSCs into an Astrocytic Phenotype

Materials and Methods

Bone marrow mesenchymal stem cells (BM-MSCs) were transduced with a GFAP-GFP reporter. The cells were then transfected with both antagomiR-138 and miR-101. The cells were viewed under a fluorescence microscope after 10 days.

Additional gene and miR arrays were used to characterize the differentiated cells.

Results

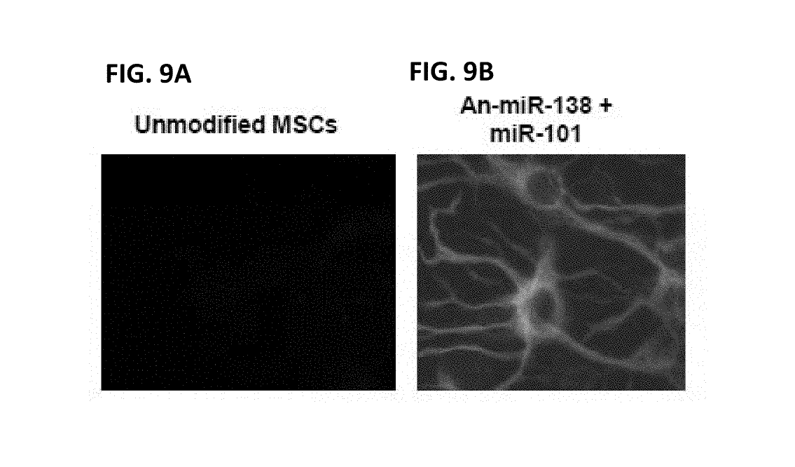

As illustrated in FIGS. 9A-B, silencing of miR-138 together with overexpression of miR-101 leads to the differentiation of MSCs into GFAP positive cells. In addition, these cells also expressed high levels of the glutamate transporters (data not shown).

miR array analysis identified the following miRs that were increased in the differentiated cells: miR-504, miR-891 and miR-874; and the following miRs that were decreased in the differentiated cells: miR-138, miR-182, miR-487, miR-214 and miR-409. Gene array analysis of the differentiated astrocytes demonstrated a decrease in a variety of genes related to osteogenic, adipogenic and chondrogenic differentiation and an increased expression of neural markers. Similarly, it was found that the differentiated astrocytes expressed high levels of NGF, IGF-1, VEGF, BDNF and GDNF. In addition, they expressed high levels of CXCR4, chemokines and IL-8 that play a role in cell migration.

Further miR array results are provided in Table 1 and Table 2 herein below.

Table 1 is a list of additional miRNAs that are up-regulated (over three fold) on differentiation of MSCs to astrocytes as described in Example 1, materials and methods as compared to non-differentiated MSCs. Table 2 is a list of additional miRNAs that are down-regulated (over three fold) on differentiation of MSCs to astrocytes as described in Example 1, materials and methods as compared to non-differentiated MSCs.

TABLE 1
miR-92ap, miR-21, miR-26a, miR-18a, miR-124, miR-99a, miR-30c,
miR-301a, miR-145-50, miR-143-3p, miR-373, miR-20b, miR-29c,
miR-29b, miR-143, let-7g, let-7a, let-7b, miR-98, miR-30a*, miR-17,
miR-1, miR-192, miR-155, miR-516-ap, miR-31, miR-181a,
miR-181b, miR-181c, miR-34-c, miR-34b*, miR-103a, miR-210, miR-
16, miR-30a, miR-31, miR-222, miR-17, miR-17*, miR-200b, miR-
200c, miR-128, miR-503, miR-424, miR-195, miR-1256, miR-203a,
miR-199, miR-93, miR-98, miR-125-a, miR-133a, miR-133b, miR-126,
miR-194, miR-346, miR-15b, miR-338-3p, miR-373, miR-205,
miR-210, miR-125, miR-1226, miR-708, miR-449, miR-422, miR-
340, miR-605, miR-522, miR-663, miR-130a, miR-130b, miR-942,
miR-572, miR-520, miR-639, miR-654, miR-519, mir-202, mir-767-
5p, mir-29a, mir-29b, mir-29c, let-7a, let-7b, let-7c, let-7d, let-7e, let-7f,
let-7g, let-7i, mir-4458, mir-4500, mir-98, mir-148a, mir-148b, mir-152,
mir-4658, mir-3662, mir-25, mir-32, mir-363, mir-367, mir-92a, mir-92b,
mir-520d-5p, mir-524-5p, mir-4724-3p, mir-1294, mir-143, mir-4770,
mir-3659, mir-145, mir-3163, mir-181a, mir-181b, mir-181c, mir-181d,
mir-4262, mir-4279, mir-144, mir-642b, mir-4742-3p, mir-3177-5p,
mir-656, mir-3121-3p, mir-106a, mir-106b, mir-17, mir-20a, mir-20b,
mir-519d, mir-93, mir-1297, mir-26a, mir-26b, mir-4465, mir-326, mir-
330-5p, mir-3927 and mir-2113.

TABLE 2
miR-204, miR-224, miR-616, miR-122, miR-299, miR-100, miR-138,
miR-140, miR-375, miR-217, miR-302, miR-372, miR-96, miR-127-3p,
miR-449, miR-135b, miR-101, miR-326, miR-324, miR-335, miR-14,
miR-16, mir-410, mir-3163, mir-148a, mir-148b, mir-152, mir-3121-3p,
mir-495, mir-203, mir-4680-3p.

### Example 4 Down-Regulation of a Synuclein in MSC Using miRNA

α-Synuclein is widely expressed in the adult brain. Mutations within the α-Synuclein gene are associated with autosomal dominant familial PD. The overexpression of the human wild-type form and the expression of α-Synuclein mutant forms exhibit a higher tendency to form insoluble aggregates and constitute the main structure of Lewy Bodies which result in increased susceptibility of neurons to oxidative stress.

Using several target prediction software tools, miR-504 was identified as a putative candidate and potential miR-504 binding sites in the 3′ UTR region of α-Synuclein were identified. Using Western blot analysis, it was found that miR-504 that induces differentiation of MSCs to astrocytes, also decreases the expression of α-Synuclein (FIG. 10).

### Example 5 Sequences

TABLE 3

| | Sequence of | Sequence of |
|---|---|---|
| Name | mature miRNA | premiRNA |
| hsa-let-7a | seq id no: 1 | seq id no: 73 |
| | | seq id no: 74 |
| | | seq id no: 75 |
| hsa-let-7b | seq id no: 2 | seq id no: 76 |
| hsa-let-7c | seq id no: 3 | seq id no: 77 |
| hsa-let-7d | seq id no: 4 | seq id no: 78 |
| hsa-let-7e | seq id no: 5 | seq id no: 79 |
| hsa-let-7f | seq id no: 6 | seq id no: 80 |
| hsa-let-7g | seq id no: 7 | seq id no: 81 |
| hsa-let-7i | seq id no: 8 | seq id no: 82 |
| hsa-mir-106a | seq id no: 9 | seq id no: 83 |
| hsa-mir-106b | seq id no: 10 | seq id no: 84 |
| hsa-mir-1294 | seq id no: 11 | seq id no: 85 |
| hsa-mir-1297 | seq id no: 12 | seq id no: 86 |
| hsa-mir-143 | seq id no: 13 | seq id no: 87 |
| hsa-mir-144 | seq id no: 14 | seq id no: 88 |
| hsa-mir-145 | seq id no: 15 | seq id no: 89 |
| hsa-mir-17 | seq id no: 16 | seq id no: 90 |
| miR-181a | seq id no: 17 | seq id no: 91 |
| miR-181a | seq id no: 18 | seq id no: 92 |
| miR-181b | seq id no: 19 | seq id no: 93 |
| miR-181b | seq id no: 20 | seq id no: 94 |
| miR-181c | seq id no: 21 | seq id no: 95 |
| hsa-mir-181d | seq id no: 22 | seq id no: 96 |
| hsa-mir-199a-3p | seq id no: 23 | seq id no: 97 |
| hsa-mir-199b-3p | seq id no: 24 | seq id no: 98 |
| hsa-mir-202 | seq id no: 25 | seq id no: 99 |
| hsa-mir-20a | seq id no: 26 | seq id no: 100 |
| hsa-mir-20b | seq id no: 27 | seq id no: 101 |
| hsa-mir-2113 | seq id no: 28 | seq id no: 102 |
| hsa-mir-25 | seq id no: 29 | seq id no: 103 |
| hsa-mir-26a | seq id no: 30 | seq id no: 104 |
| | seq id no: 31 | seq id no: 105 |
| hsa-mir-26b | seq id no: 32 | seq id no: 106 |
| hsa-mir-29a | seq id no: 33 | seq id no: 107 |
| hsa-mir-29b | seq id no: 34 | seq id no: 108 |
| | | seq id no: 109 |
| hsa-mir-29c | seq id no: 35 | seq id no: 110 |
| hsa-mir-3129-5p | seq id no: 36 | seq id no: 111 |
| hsa-mir-3177-5p | seq id no: 37 | seq id no: 112 |
| hsa-mir-32 | seq id no: 38 | seq id no: 113 |
| hsa-mir-326 | seq id no: 39 | seq id no: 114 |
| hsa-mir-330-5p | seq id no: 40 | seq id no: 115 |
| hsa-mir-363 | seq id no: 41 | seq id no: 116 |
| hsa-mir-3659 | seq id no: 42 | seq id no: 117 |
| hsa-mir-3662 | seq id no: 43 | seq id no: 118 |
| hsa-mir-367 | seq id no: 44 | seq id no: 119 |
| hsa-mir-372 | seq id no: 45 | seq id no: 120 |
| hsa-mir-373 | seq id no: 46 | seq id no: 121 |
| hsa-mir-3927 | seq id no: 47 | seq id no: 122 |
| hsa-mir-4262 | seq id no: 48 | seq id no: 123 |
| hsa-mir-4279 | seq id no: 49 | seq id no: 124 |
| hsa-mir-4458 | seq id no: 50 | seq id no: 125 |
| hsa-mir-4465 | seq id no: 51 | seq id no: 126 |
| hsa-mir-4500 | seq id no: 52 | seq id no: 127 |
| hsa-mir-4658 | seq id no: 53 | seq id no: 128 |
| hsa-mir-4724-3p | seq id no: 54 | seq id no: 129 |
| hsa-mir-4742-3p | seq id no: 55 | seq id no: 130 |
| hsa-mir-4770 | seq id no: 56 | seq id no: 131 |
| hsa-mir-519d | seq id no: 57 | seq id no: 132 |
| hsa-mir-520a-3p | seq id no: 58 | seq id no: 133 |
| hsa-mir-520b | seq id no: 59 | seq id no: 134 |
| hsa-mir-520c-3p | seq id no: 60 | seq id no: 135 |
| hsa-mir-520d-3p | seq id no: 61 | seq id no: 136 |
| hsa-mir-520d-5p | seq id no: 62 | seq id no: 137 |
| hsa-mir-520e | seq id no: 63 | seq id no: 138 |
| hsa-mir-524-5p | seq id no: 64 | seq id no: 139 |
| hsa-mir-642b | seq id no: 65 | seq id no: 140 |
| hsa-mir-656 | seq id no: 66 | seq id no: 141 |
| hsa-mir-767-5p | seq id no: 67 | seq id no: 142 |
| hsa-mir-92a | seq id no: 68 | seq id no: 143 |
| | seq id no: 69 | seq id no: 144 |
| hsa-mir-92b | seq id no: 70 | seq id no: 145 |
| hsa-mir-93 | seq id no: 71 | seq id no: 146 |
| hsa-mir-98 | seq id no: 72 | seq id no: 147 |

TABLE 4

| | | Sequence of |
|---|---|---|
| Name | Sequence of mature | premiRNA |
| hsa-mir-410 | seq id no: 148 | seq id no: 156 |
| hsa-mir-3163 | seq id no: 149 | seq id no: 157 |
| hsa-mir-148a | seq id no: 150 | seq id no: 158 |
| hsa-mir-148b | seq id no: 151 | seq id no: 159 |
| hsa-mir-152 | seq id no: 152 | seq id no: 160 |
| hsa-mir-3121-3p | seq id no: 153 | seq id no: 161 |
| hsa-mir-495 | seq id no: 154 | seq id no: 162 |
| hsa-mir-4680-3p | seq id no: 155 | seq id no: 163 |

TABLE 5

| | Sequence of | | Sequence of |
|---|---|---|---|
| Name | mature | PMIR id | premiRNA |
| miR-92ap | seq id no: 164 | MI0000093 | seq id no: 269 |
| | seq id no: 165 | MI0000094 | seq id no: 270 |
| miR-21 | seq id no: 166 | MI0000077 | seq id no: 271 |
| miR-26a 5P | seq id no: 167 | MI0000083 | seq id no: 272 |
| | seq id no: 168 | MI0000750 | seq id no: 273 |
| miR-18a | seq id no: 169 | MI0000072 | seq id no: 274 |
| miR-124 | seq id no: 170 | MI0000445 | seq id no: 275 |
| | seq id no: 171 | MI0000443 | seq id no: 276 |
| | seq id no: 172 | MI0000444 | seq id no: 277 |
| miR-99a | seq id no: 173 | MI0000101 | seq id no: 278 |
| miR-30c | seq id no: 174 | MI0000736 | seq id no: 279 |
| | | MI0000254 | seq id no: 280 |
| miR-301a 3P | seq id no: 175 | MI0000745 | seq id no: 281 |
| miR-145-50 | seq id no: 176 | MI0000461 | seq id no: 282 |
| miR-143-3p | seq id no: 177 | MI0000459 | seq id no: 283 |
| miR-373 3P | seq id no: 178 | MI0000781 | seq id no: 284 |
| miR-20b | seq id no: 179 | MI0001519 | seq id no: 285 |
| miR-29c 3P | seq id no: 180 | MI0000735 | seq id no: 286 |
| miR-29b 3P | seq id no: 181 | MI0000105 | seq id no: 287 |
| miR-143 | | MI0000107 | seq id no: 288 |
| let-7g | seq id no: 182 | MI0000433 | seq id no: 289 |
| let-7a | seq id no: 183 | MI0000060 | seq id no: 290 |
| | | MI0000061 | seq id no: 291 |
| | | MI0000062 | seq id no: 292 |
| let-7b | seq id no: 184 | MI0000063 | seq id no: 293 |
| miR-98 | seq id no: 185 | MI0000100 | seq id no: 294 |
| miR-30a* | seq id no: 186 | MI0000088 | seq id no: 295 |
| miR-17 | seq id no: 187 | MI0000071 | seq id no: 296 |
| miR-1-1 | seq id no: 188 | MI0000651 | seq id no: 297 |
| miR-1-2 | seq id no: 189 | MI0000437 | seq id no: 298 |
| miR-192 | seq id no: 190 | MI0000234 | seq id no: 299 |
| miR-155 | seq id no: 191 | MI0000681 | seq id no: 300 |
| miR-516-ap a1- | seq id no: 192 | MI0003180 | seq id no: 301 |
| 5p- | | | |
| a2-3p- | seq id no: 193 | MI0003181 | seq id no: 302 |
| miR-31 | seq id no: 194 | MI0000089 | seq id no: 303 |
| miR-181a | seq id no: 195 | MI0000289 | seq id no: 304 |
| | seq id no: 196 | MI0000269 | seq id no: 305 |
| miR-181b | seq id no: 197 | MI0000270 | seq id no: 306 |
| | seq id no: 198 | MI0000683 | seq id no: 307 |
| miR-181c | seq id no: 199 | MI0000271 | seq id no: 308 |
| miR-34-c | seq id no: 200 | MI0000743 | seq id no: 309 |
| miR-34b* | seq id no: 201 | MI0000742 | seq id no: 310 |
| miR-103a | seq id no: 202 | MI0000109 | seq id no: 311 |
| | seq id no: 203 | MI0000108 | seq id no: 312 |
| miR-210 | seq id no: 204 | MI0000286 | seq id no: 313 |
| miR-16 | seq id no: 205 | MI0000070 | seq id no: 314 |
| | seq id no: 206 | MI0000115 | seq id no: 315 |
| miR-30a | seq id no: 207 | MI0000088 | seq id no: 316 |
| miR-31 | seq id no: 208 | MI0000089 | seq id no: 317 |
| miR-222 | seq id no: 209 | MI0000299 | seq id no: 318 |
| miR-17 | seq id no: 210 | MI0000071 | seq id no: 319 |
| miR-17* | seq id no: 211 | MI0000071 | seq id no: 320 |
| miR-200b | seq id no: 212 | MI0000342 | seq id no: 321 |
| miR-200c | seq id no: 213 | MI0000650 | seq id no: 322 |
| miR-128 | seq id no: 214 | MI0000447 | seq id no: 323 |
| | | MI0000727 | seq id no: 324 |
| miR-503 | seq id no: 215 | MI0003188 | seq id no: 325 |
| miR-424 | seq id no: 216 | MI0001446 | seq id no: 326 |
| miR-195 | seq id no: 217 | MI0000489 | seq id no: 327 |
| miR-1256 | seq id no: 218 | MI0006390 | seq id no: 328 |
| miR-203a | seq id no: 219 | MI0000283 | seq id no: 329 |
| miR-199 | | | |
| hsa-miR-199a- | seq id no: 220 | MI0000242 | seq id no: 330 |
| 3p_st | | | |
| hsa-miR-199a- | seq id no: 221 | MI0000242 | seq id no: 331 |
| 5p_st | | | |
| hsa-miR-199b- | seq id no: 222 | MI0000282 | seq id no: 332 |
| 3p_st | | | |
| miR-93 | seq id no: 223 | MI0000095 | seq id no: 333 |
| miR-98 | seq id no: 224 | MI0000100 | seq id no: 334 |
| miR-125-a | seq id no: 225 | MI0000469 | seq id no: 335 |
| miR-133a | seq id no: 226 | MI0000450 | seq id no: 336 |
| | | MI0000451 | seq id no: 337 |
| miR-133b | seq id no: 227 | MI0000822 | seq id no: 338 |
| miR-126 | seq id no: 228 | MI0000471 | seq id no: 339 |
| miR-194 | seq id no: 229 | MI0000488 | seq id no: 340 |
| | | MI0000732 | seq id no: 341 |
| miR-346 | seq id no: 230 | MI0000826 | seq id no: 342 |
| miR-15b | seq id no: 231 | MI0000438 | seq id no: 343 |
| miR-338-3p | seq id no: 232 | MI0000814 | seq id no: 344 |
| miR-373 | | | |
| miR-205 | seq id no: 233 | MI0000285 | seq id no: 345 |
| miR-210 | | | |
| miR-125 | | | |
| miR-1226 | seq id no: 234 | MI0006313 | seq id no: 346 |
| miR-708 | seq id no: 235 | MI0005543 | seq id no: 347 |
| miR-449 | seq id no: 236 | MI0001648 | seq id no: 348 |
| miR-422 | seq id no: 237 | MI0001444 | seq id no: 349 |
| miR-340 | seq id no: 238 | MI0000802 | seq id no: 350 |
| miR-605 | seq id no: 239 | MI0003618 | seq id no: 351 |
| miR-522 | seq id no: 240 | MI0003177 | seq id no: 352 |
| miR-663 | seq id no: 241 | MI0003672 | seq id no: 353 |
| miR-130a | seq id no: 242 | MI0000448 | seq id no: 354 |
| miR-130b | seq id no: 243 | MI0000748 | seq id no: 355 |
| miR-942 | seq id no: 244 | MI0005767 | seq id no: 356 |
| miR-572 | seq id no: 245 | MI0003579 | seq id no: 357 |
| miR-520 | | | |
| miR-639 | seq id no: 246 | MI0003654 | seq id no: 358 |
| miR-654 | seq id no: 247 | MI0003676 | seq id no: 359 |
| miR-519 | | | |
| miR-204 | seq id no: 248 | MI0000284 | |
| miR-224 | seq id no: 249 | MI0000301 | seq id no: 360 |
| miR-616 | seq id no: 250 | MI0003629 | seq id no: 361 |
| miR-122 | seq id no: 251 | MI0000442 | seq id no: 362 |
| miR-299 3p- | seq id no: 252 | MI0000744 | seq id no: 363 |
| 5p- | seq id no: 253 | | seq id no: 364 |
| miR-100 | seq id no: 254 | MI0000102 | |
| miR-138 | seq id no: 255 | MI0000476 | seq id no: 365 |
| miR-140 | seq id no: 256 | MI0000456 | seq id no: 366 |
| miR-375 | seq id no: 257 | MI0000783 | seq id no: 367 |
| miR-217 | seq id no: 258 | MI0000293 | seq id no: 368 |
| miR-302 | | | seq id no: 369 |
| miR-372 | seq id no: 259 | MI0000780 | |
| miR-96 | seq id no: 260 | MI0000098 | seq id no: 370 |
| miR-127-3p | seq id no: 261 | MI0000472 | seq id no: 371 |
| miR-449 | | | seq id no: 372 |
| miR-135b | seq id no: 262 | MI0000810 | |
| miR-101 | seq id no: 263 | MI0000103 | seq id no: 373 |
| | | MI0000739 | seq id no: 374 |
| miR-326 | seq id no: 264 | MI0000808 | seq id no: 375 |
| miR-3245p- | seq id no: 265 | MI0000813 | seq id no: 376 |
| 3p- | seq id no: 266 | MI0000813 | seq id no: 377 |
| miR-335 | seq id no: 267 | MI0000816 | seq id no: 378 |
| miR-141 | seq id no: 268 | MI0000457 | seq id no: 379 |

TABLE 6

| | Sequence of | |
|---|---|---|
| | mature | Sequence of |
| Name | miRNA | premiRNA |
| miR-1275 | seq id no: | seq id no: |
| | 381 | 414 |
| miR-891a | seq id no: | seq id no: |
| | 382 | 415 |
| miR-154 | seq id no: | seq id no: |
| | 383 | 416 |
| miR-1202 | seq id no: | seq id no: |
| | 384 | 417 |
| miR-572 | seq id no: | seq id no: |
| | 385 | 418 |
| miR-935a | seq id no: | seq id no: |
| | 386 | 419 |
| miR-4317 | seq id no: | seq id no: |
| | 387 | 420 |
| miR-153 | seq id no: | seq id no: |
| | 388 | 421 |
| | | seq id no: |
| | | 422 |
| miR-4288 | seq id no: | seq id no: |
| | 389 | 423 |
| miR-409-5p | seq id no: | seq id no: |
| | 390 | 424 |
| miR-193a-5p | seq id no: | seq id no: |
| | 391 | 425 |
| miR-648 | seq id no: | seq id no: |
| | 392 | 426 |
| miR-368 | | |
| miR-365 | seq id no: | seq id no: |
| | 393 | 427 |
| miR-500 | seq id no: | seq id no: |
| | 394 | 428 |
| miR-491 | seq id no: | seq id no: |
| | 395 | 429 |
| hsa-miR-199a- | seq id no: | seq id no: |
| 3p_st | 396 | 430 |
| | seq id no: | seq id no: |
| | 397 | 431 |
| hsa-miR-199a- | seq id no: | seq id no: |
| 5p_st | 398 | 432 |
| | seq id no: | seq id no: |
| | 399 | 433 |
| miR-2113 | seq id no: | seq id no: |
| | 400 | 434 |
| miR-372 | seq id no: | seq id no: |
| | 401 | 435 |
| miR-373 | seq id no: | seq id no: |
| | 402 | 436 |
| miR-942 | seq id no: | seq id no: |
| | 403 | 437 |
| miR-1293 | seq id no: | seq id no: |
| | 404 | 438 |
| miR-18 | seq id no: | seq id no: |
| | 405 | 439 |
| miR-1182 | seq id no: | seq id no: |
| | 406 | 440 |
| miR-1185 | seq id no: | seq id no: |
| | 407 | 441 |
| | | seq id no: |
| | | 442 |
| miR-1276 | seq id no: | seq id no: |
| | 408 | 443 |
| miR-193b | seq id no: | seq id no: |
| | 409 | 444 |
| miR-1238 | seq id no: | seq id no: |
| | 410 | 445 |
| miR-889 | seq id no: | seq id no: |
| | 411 | 446 |
| miR-370 | seq id no: | seq id no: |
| | 412 | 447 |
| miR-548-d1 | seq id no: | seq id no: |
| | 413 | 448 |

TABLE 7

| | Sequence |
|---|---|
| | of mature |
| Name | miRNA |
| hsa-miR-20b | seq id no: |
| | 449 |
| hsa-miR-18 | seq id no: |
| | 450 |
| hsa-miR-17- | seq id no: |
| 5p | 451 |
| hsa-miR-141 | seq id no: |
| | 452 |
| hsa-miR- | seq id no: |
| 302b | 453 |
| hsa-miR-101 | seq id no: |
| | 454 |
| hsa-miR-126 | seq id no: |
| | 455 |
| hsa-miR- | seq id no: |
| 146a | 456 |
| hsa-miR- | seq id no: |
| 146b | 457 |
| hsa-miR-26 | seq id no: |
| | 458 |
| hsa-miR-29 | seq id no: |
| | 459 |
| hsa-miR-132 | seq id no: |
| | 460 |
| hsa-miR-9 | seq id no: |
| | 461 |
| hsa-miR-146 | seq id no: |
| | 462 |
| hsa-miR-10b | seq id no: |
| | 463 |
| hsa-miR- | seq id no: |
| 222 | 464 |
| hsa-miR- | seq id no: |
| 193b | 465 |
| hsa-miR- | seq id no: |
| 221 | 466 |
| hsa-miR- | seq id no: |
| 135a | 467 |
| hsa-miR- | seq id no: |
| 149 | 468 |
| hsa-miR- | seq id no: |
| 199a | 469 |
| hsa-miR- | seq id no: |
| 302a | 470 |
| hsa-miR- | seq id no: |
| 302c | 471 |
| hsa-miR- | seq id no: |
| 302d | 472 |
| hsa-miR- | seq id no: |
| 369-3p | 473 |
| hsa-miR- | seq id no: |
| 370 | 474 |
| hsa-miR- | seq id no: |
| let7a | 475 |
| hsa-miR- | seq id no: |
| let7b | 476 |
| hsa-miR- | seq id no: |
| 10b | 477 |
| hsa-miR- | seq id no: |
| 23a | 478 |
| hsa-miR- | seq id no: |
| 23b | 479 |
| hsa-miR-32 | seq id no: |
| | 480 |

### Example 6: Combined miR-504 and Anti-miR-302 Differentiated MSCs for Treating Parkinson's Disease

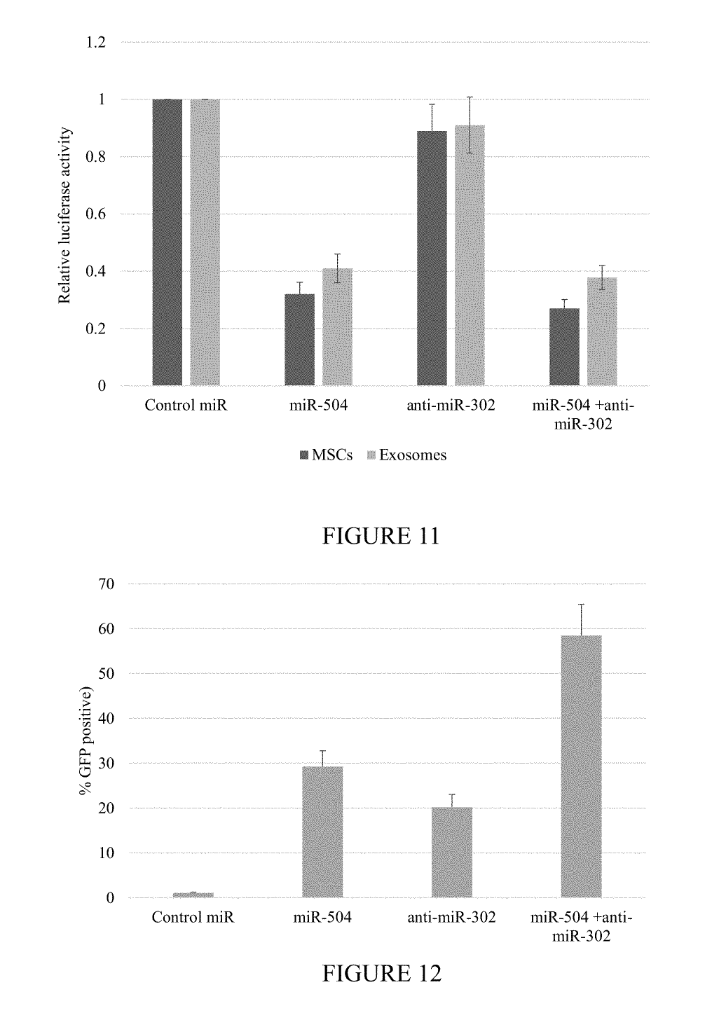

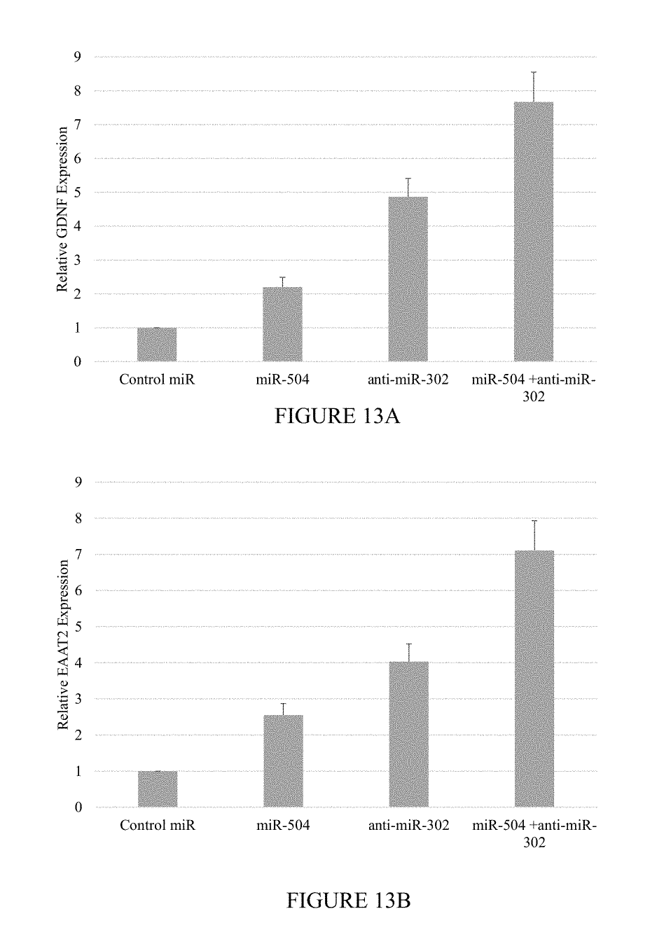

As shown hereinabove, miR-504 is effective in reducing α-synuclein (SNCA) levels in neuronal cells in culture (FIG. 10). SNCA is known to play a major role in the pathogenesis of neurodegenerative diseases, such as Parkinson's disease (PD), where its overexpression is thought to contribute to the pathology. Also, as reported hereinabove (Examples 3-4), ectopic expression of miR-504 in MSCs induces conversion of the MSC to an astrocytic phenotype. Specifically, transfection of MSCs with miR-504 increased expression of GFAP (FIG. 12), as well as GDNF (FIG. 13A) and EAAT2 (FIG. 13B). Astrocytes themselves are helpful in treating a number of neurodegenerative diseases, including those that are characterized by SNCA expression. In order to increase the astrocyte-like phenotype of the MSCs transfected with miR-504, the cells were further transfected with an antagomir against miR-302. MSCs transfected with either miR-504, or anti-miR-302 took on an astrocyte phenotype and expressed GFAP according to a reporter assay using the GFAP promoter (FIG. 12). Unexpectedly, the combination of miR-504 and anti-miR-302 increased GFAP expression to even greater levels (FIG. 12), a result that was not observed when anti-miR-138 (another anti-miR that induces an astrocyte phenotype) was combined with miR-504. The synergistic effect of combining miR-504 and anti-miR-302 was also seen in increased GDNF (FIG. 13A) and EAAT2 (FIG. 13B) expression. MSCs expressing miR-504 reduced SNCA expression by over 60% (FIG. 11). MSCs expression anti-miR-302 had a negligible effect on SNCA, and the combination of the two was slightly better than miR-504 alone (FIG. 11). Further, exosomes, extracellular vesicles isolated from the MSCs, had nearly as strong an effect (FIG. 11). This combination of miR-504 and anti-miR-302 is doubly effective because it had a stronger astrocytic differentiation effect, and thus a stronger therapeutic effect, since GDNF is essential for the survival of dopaminergic neurons. Thus, MSCs transfected with miR-504, or a combination of miR-504 and anti-miR-302, and/or exosomes derived from those cells, are a novel therapeutic approach for treating neurodegenerative diseases with increased SNCA and specifically Parkinson's disease.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An isolated population of genetically modified mesenchymal stem cells (MSCs) differentiated toward an astrocyte phenotype wherein each MSC comprises an exogenous microRNA (miR)-504, wherein at least 50% of the MSCs express glial fibrillary acidic protein.

2. The isolated population of claim 1, wherein at least 50% of the population of MSCs differentiated toward an astrocytic phenotype is further identified by expression of a marker selected from the group consisting of GDNF, protein S100, glutamine synthetase, excitatory amino acid transporter 1 (EAAT1) and EAAT2.

3. The isolated population of claim 1, wherein the at least 50% of the population of MSCs differentiated toward an astrocytic phenotype is further identified by astrocytic morphology.

4. The isolated population of claim 1, wherein said MSCs are isolated from a tissue selected from the group consisting of bone marrow, adipose tissue, placenta, cord blood and umbilical cord.

5. The isolated population of claim 1, wherein said each MSC further comprises an antagomir or RNA oligonucleotide that hybridizes to an inhibits an endogenous miR-302.

6. A method of generating the isolated population of claim 1, the method comprising introducing and expressing in MSCs an exogenous miR-504, thereby generating an isolated population of genetically modified MSCs differentiated toward and astrocyte phenotype.

7. The method of claim 6, wherein said introducing and expressing comprises transfecting said MSCs with an expression vector which comprises a polynucleotide sequence which encodes a pre-miRNA of said miR-504 or a polynucleotide sequence which encodes said miR-504.

8. The method of claim 6, further comprising analyzing expression of at least one marker selected from the group consisting of GDNF, S100, glutamine synthetase, excitatory amino acid transporter 1 and EAAT2 following said generating.

9. The method of claim 6, further comprising incubating said MSCs in a differentiation medium comprising at least one agent selected from the group consisting of platelet derived growth factor (PDGF), neuregulin, fibroblast growth factor 2 (FGF-b) and a c-AMP inducing agent following, prior to or concomitant with said expressing.

10. The method of claim 6, further comprising introducing and expressing in said MSCs an antagomir or RNA oligonucleotide that hybridizes to an inhibits endogenous miR-302.

11. A pharmaceutical composition comprising the isolated population of claim 1 and a pharmaceutically acceptable carrier.

12. A pharmaceutical composition comprising the isolated population of claim 5 and a pharmaceutically acceptable carrier.

13. A method of decreasing expression of α-synuclein in a target cell, the method comprising contacting said target cell with the isolated population of claim 1.

14. A method of decreasing expression of α-synuclein in a target cell, the method comprising contacting said target cell with the isolated population of claim 5.

15. A method of treating Parkinson's disease in a subject in need thereof, comprising administer to said subject the pharmaceutical composition of claim 11.

16. The method of claim 15, wherein said composition comprises a therapeutically effective amount of MSCs.

17. The method of claim 15, wherein said MSCs are autologous, non-autologous or semi-autologous to said subject.

18. A method of treating Parkinson's disease in a subject in need thereof, comprising administer to said subject the pharmaceutical composition of claim 12.

19. The method of claim 18, wherein said composition comprises a therapeutically effective amount of MSCs.

20. The method of claim 18, wherein said MSCs are autologous, non-autologous or semi-autologous to said subject.


SEQUENCE LISTING (501 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
agagguaguagguugcauaguu

SEQ ID NO: 5 - RNA, 22 nt, Homo sapiens
ugagguaggagguuguauaguu

SEQ ID NO: 6 - RNA, 22 nt, Homo sapiens
ugagguaguagauuguauaguu

SEQ ID NO: 7 - RNA, 22 nt, Homo sapiens
ugagguaguaguuuguacaguu

SEQ ID NO: 8 - RNA, 22 nt, Homo sapiens
ugagguaguaguuugugcuguu

SEQ ID NO: 9 - RNA, 23 nt, Homo sapiens
aaaagugcuuacagugcagguag

SEQ ID NO: 10 - RNA, 21 nt, Homo sapiens
uaaagugcugacagugcagau

SEQ ID NO: 11 - RNA, 22 nt, Homo sapiens
ugugagguuggcauuguugucu

SEQ ID NO: 12 - RNA, 17 nt, Homo sapiens
uucaaguaauucaggug

SEQ ID NO: 13 - RNA, 22 nt, Homo sapiens
ggugcagugcugcaucucuggu

SEQ ID NO: 14 - RNA, 20 nt, Homo sapiens
uacaguauagaugauguacu

SEQ ID NO: 15 - RNA, 23 nt, Homo sapiens
guccaguuuucccaggaaucccu

SEQ ID NO: 16 - RNA, 23 nt, Homo sapiens
caaagugcuuacagugcagguag

SEQ ID NO: 17 - RNA, 23 nt, Homo sapiens
aacauucaacgcugucggugagu

SEQ ID NO: 18 - RNA, 23 nt, Homo sapiens
aacauucaacgcugucggugagu

SEQ ID NO: 19 - RNA, 23 nt, Homo sapiens
aacauucauugcugucggugggu

SEQ ID NO: 20 - RNA, 23 nt, Homo sapiens
aacauucauugcugucggugggu

SEQ ID NO: 21 - RNA, 22 nt, Homo sapiens
aacauucaaccugucggugagu

SEQ ID NO: 22 - RNA, 23 nt, Homo sapiens
aacauucauuguugucggugggu

SEQ ID NO: 23 - RNA, 22 nt, Homo sapiens
acaguagucugcacauugguua

SEQ ID NO: 24 - RNA, 22 nt, Homo sapiens
acaguagucugcacauugguua

SEQ ID NO: 25 - RNA, 20 nt, Homo sapiens
agagguauagggcaugggaa

SEQ ID NO: 26 - RNA, 23 nt, Homo sapiens
uaaagugcuuauagugcagguag

SEQ ID NO: 27 - RNA, 23 nt, Homo sapiens
caaagugcucauagugcagguag

SEQ ID NO: 28 - RNA, 21 nt, Homo sapiens
auuugugcuuggcucugucac

SEQ ID NO: 29 - RNA, 22 nt, Homo sapiens
cauugcacuugucucggucuga

SEQ ID NO: 30 - RNA, 22 nt, Homo sapiens
uucaaguaauccaggauaggcu

SEQ ID NO: 31 - RNA, 22 nt, Homo sapiens
uucaaguaauccaggauaggcu

SEQ ID NO: 32 - RNA, 21 nt, Homo sapiens
uucaaguaauucaggauaggu

SEQ ID NO: 33 - RNA, 22 nt, Homo sapiens
uagcaccaucugaaaucgguua

SEQ ID NO: 34 - RNA, 23 nt, Homo sapiens
uagcaccauuugaaaucaguguu

SEQ ID NO: 35 - RNA, 22 nt, Homo sapiens
uagcaccauuugaaaucgguua

SEQ ID NO: 36 - RNA, 22 nt, Homo sapiens
gcaguaguguagagauugguuu

SEQ ID NO: 37 - RNA, 23 nt, Homo sapiens
uguguacacacgugccaggcgcu

SEQ ID NO: 38 - RNA, 22 nt, Homo sapiens
uauugcacauuacuaaguugca

SEQ ID NO: 39 - RNA, 20 nt, Homo sapiens
ccucugggcccuuccuccag

SEQ ID NO: 40 - RNA, 23 nt, Homo sapiens
gcaaagcacacggccugcagaga

SEQ ID NO: 41 - RNA, 22 nt, Homo sapiens
aauugcacgguauccaucugua

SEQ ID NO: 42 - RNA, 21 nt, Homo sapiens
ugaguguugucuacgagggca

SEQ ID NO: 43 - RNA, 24 nt, Homo sapiens
gaaaaugaugaguagugacugaug

SEQ ID NO: 44 - RNA, 22 nt, Homo sapiens
aauugcacuuuagcaaugguga

SEQ ID NO: 45 - RNA, 23 nt, Homo sapiens
aaagugcugcgacauuugagcgu

SEQ ID NO: 46 - RNA, 23 nt, Homo sapiens
gaagugcuucgauuuuggggugu

SEQ ID NO: 47 - RNA, 22 nt, Homo sapiens
cagguagauauuugauaggcau

SEQ ID NO: 48 - RNA, 17 nt, Homo sapiens
gacauucagacuaccug

SEQ ID NO: 49 - RNA, 16 nt, Homo sapiens
cucuccucccggcuuc

SEQ ID NO: 50 - RNA, 19 nt, Homo sapiens
agagguagguguggaagaa

SEQ ID NO: 51 - RNA, 22 nt, Homo sapiens
cucaaguagucugaccagggga

SEQ ID NO: 52 - RNA, 17 nt, Homo sapiens
ugagguaguaguuucuu

SEQ ID NO: 53 - RNA, 23 nt, Homo sapiens
gugaguguggauccuggaggaau

SEQ ID NO: 54 - RNA, 21 nt, Homo sapiens
guaccuucugguucagcuagu

SEQ ID NO: 55 - RNA, 23 nt, Homo sapiens
ucuguauucuccuuugccugcag

SEQ ID NO: 56 - RNA, 18 nt, Homo sapiens
ugagaugacacuguagcu

SEQ ID NO: 57 - RNA, 22 nt, Homo sapiens
caaagugccucccuuuagagug

SEQ ID NO: 58 - RNA, 22 nt, Homo sapiens
aaagugcuucccuuuggacugu

SEQ ID NO: 59 - RNA, 21 nt, Homo sapiens
aaagugcuuccuuuuagaggg

SEQ ID NO: 60 - RNA, 22 nt, Homo sapiens
aaagugcuuccuuuuagagggu

SEQ ID NO: 61 - RNA, 22 nt, Homo sapiens
aaagugcuucucuuuggugggu

SEQ ID NO: 62 - RNA, 20 nt, Homo sapiens
cuacaaagggaagcccuuuc

SEQ ID NO: 63 - RNA, 21 nt, Homo sapiens
aaagugcuuccuuuuugaggg

SEQ ID NO: 64 - RNA, 22 nt, Homo sapiens
cuacaaagggaagcacuuucuc

SEQ ID NO: 65 - RNA, 22 nt, Homo sapiens
agacacauuuggagagggaccc

SEQ ID NO: 66 - RNA, 21 nt, Homo sapiens
aauauuauacagucaaccucu

SEQ ID NO: 67 - RNA, 23 nt, Homo sapiens
ugcaccaugguugucugagcaug

SEQ ID NO: 68 - RNA, 22 nt, Homo sapiens
uauugcacuugucccggccugu

SEQ ID NO: 69 - RNA, 22 nt, Homo sapiens
uauugcacuugucccggccugu

SEQ ID NO: 70 - RNA, 22 nt, Homo sapiens
uauugcacucgucccggccucc

SEQ ID NO: 71 - RNA, 23 nt, Homo sapiens
caaagugcuguucgugcagguag

SEQ ID NO: 72 - RNA, 22 nt, Homo sapiens
ugagguaguaaguuguauuguu

SEQ ID NO: 73 - RNA, 80 nt, Homo sapiens
ugggaugagguaguagguuguauaguuuuagggucacacccaccacugggagauaacuauacaaucuacugucuuuccua

SEQ ID NO: 74 - RNA, 72 nt, Homo sapiens
agguugagguaguagguuguauaguuuagaauuacaucaagggagauaacuguacagccuccuagcuuuccu

SEQ ID NO: 75 - RNA, 74 nt, Homo sapiens
gggugagguaguagguuguauaguuuggggcucugcccugcuaugggauaacuauacaaucuacugucuuuccu

SEQ ID NO: 76 - RNA, 83 nt, Homo sapiens
cggggugagguaguagguugugugguuucagggcagugauguugccccucggaagauaacuauacaaccuacugccuucccug

SEQ ID NO: 77 - RNA, 84 nt, Homo sapiens
gcauccggguugagguaguagguuguaugguuuagaguuacacccugggaguuaacuguacaaccuucuagcuuuccuuggagc

SEQ ID NO: 78 - RNA, 87 nt, Homo sapiens
ccuaggaagagguaguagguugcauaguuuuagggcagggauuuugcccacaaggagguaacuauacgaccugcugccuuucuuagg

SEQ ID NO: 79 - RNA, 79 nt, Homo sapiens
cccgggcugagguaggagguuguauaguugaggaggacacccaaggagaucacuauacggccuccuagcuuuccccagg

SEQ ID NO: 80 - RNA, 87 nt, Homo sapiens
ucagagugagguaguagauuguauaguugugggguagugauuuuacccuguucaggagauaacuauacaaucuauugccuucccuga

SEQ ID NO: 81 - RNA, 84 nt, Homo sapiens
aggcugagguaguaguuuguacaguuugagggucuaugauaccacccgguacaggagauaacuguacaggccacugccuugcca

SEQ ID NO: 82 - RNA, 84 nt, Homo sapiens
cuggcugagguaguaguuugugcuguuggucggguugugacauugcccgcuguggagauaacugcgcaagcuacugccuugcua

SEQ ID NO: 83 - RNA, 81 nt, Homo sapiens
ccuuggccauguaaaagugcuuacagugcagguagcuuuuugagaucuacugcaauguaagcacuucuuacauuaccaugg

SEQ ID NO: 84 - RNA, 82 nt, Homo sapiens
ccugccggggcuaaagugcugacagugcagauagugguccucuccgugcuaccgcacuguggguacuugcugcuccagcagg

SEQ ID NO: 85 - RNA, 142 nt, Homo sapiens
caccuaaugugugccaagaucuguucauuuaugaucucaccgaguccugugagguuggcauuguugucuggcauugucugauauacaacagugccaaccucacaggacucagugaggugaaacugaggauuaggaaggugua

SEQ ID NO: 86 - RNA, 77 nt, Homo sapiens
uguuuaucucuaggguugaucuauuagaauuacuuaucugagccaaaguaauucaaguaauucagguguagugaaac

SEQ ID NO: 87 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 88 - RNA, 86 nt, Homo sapiens
uggggcccuggcugggauaucaucauauacuguaaguuugcgaugagacacuacaguauagaugauguacuaguccgggcaccccc

SEQ ID NO: 89 - RNA, 88 nt, Homo sapiens
caccuuguccucacgguccaguuuucccaggaaucccuuagaugcuaagauggggauuccuggaaauacuguucuugaggucaugguu

SEQ ID NO: 90 - RNA, 84 nt, Homo sapiens
gucagaauaaugucaaagugcuuacagugcagguagugauaugugcaucuacugcagugaaggcacuuguagcauuauggugac

SEQ ID NO: 91 - RNA, 110 nt, Homo sapiens
ugaguuuugagguugcuucagugaacauucaacgcugucggugaguuuggaauuaaaaucaaaaccaucgaccguugauuguacccuauggcuaaccaucaucuacucca

SEQ ID NO: 92 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 93 - RNA, 110 nt, Homo sapiens
ccugugcagagauuauuuuuuaaaaggucacaaucaacauucauugcugucgguggguugaacuguguggacaagcucacugaacaaugaaugcaacuguggccccgcuu

SEQ ID NO: 94 - RNA, 89 nt, Homo sapiens
cugauggcugcacucaacauucauugcugucgguggguuugagucugaaucaacucacugaucaaugaaugcaaacugcggaccaaaca

SEQ ID NO: 95 - RNA, 110 nt, Homo sapiens
cggaaaauuugccaaggguuugggggaacauucaaccugucggugaguuugggcagcucaggcaaaccaucgaccguugaguggacccugaggccuggaauugccauccu

SEQ ID NO: 96 - RNA, 137 nt, Homo sapiens
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 97 - RNA, 71 nt, Homo sapiens
gccaacccaguguucagacuaccuguucaggaggcucucaauguguacaguagucugcacauugguuaggc

SEQ ID NO: 98 - RNA, 110 nt, Homo sapiens
ccagaggacaccuccacuccgucuacccaguguuuagacuaucuguucaggacucccaaauuguacaguagucugcacauugguuaggcugggcuggguuagacccucgg

SEQ ID NO: 99 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 100 - RNA, 71 nt, Homo sapiens
guagcacuaaagugcuuauagugcagguaguguuuaguuaucuacugcauuaugagcacuuaaaguacugc

SEQ ID NO: 101 - RNA, 69 nt, Homo sapiens
aguaccaaagugcucauagugcagguaguuuuggcaugacucuacuguaguaugggcacuuccaguacu

SEQ ID NO: 102 - RNA, 91 nt, Homo sapiens
uuuucaaagcaaugugugacagguacagggacaaaucccguuaauaaguaagaggauuugugcuuggcucugucacaugccacuuugaaaa

SEQ ID NO: 103 - RNA, 84 nt, Homo sapiens
ggccaguguugagaggcggagacuugggcaauugcuggacgcugcccugggcauugcacuugucucggucugacagugccggcc

SEQ ID NO: 104 - RNA, 77 nt, Homo sapiens
guggccucguucaaguaauccaggauaggcugugcaggucccaaugggccuauucuugguuacuugcacggggacgc

SEQ ID NO: 105 - RNA, 84 nt, Homo sapiens
ggcuguggcuggauucaaguaauccaggauaggcuguuuccaucugugaggccuauucuugauuacuuguuucuggaggcagcu

SEQ ID NO: 106 - RNA, 77 nt, Homo sapiens
ccgggacccaguucaaguaauucaggauagguugugugcuguccagccuguucuccauuacuuggcucggggaccgg

SEQ ID NO: 107 - RNA, 64 nt, Homo sapiens
augacugauuucuuuugguguucagagucaauauaauuuucuagcaccaucugaaaucgguuau

SEQ ID NO: 108 - RNA, 81 nt, Homo sapiens
cuucaggaagcugguuucauauggugguuuagauuuaaauagugauugucuagcaccauuugaaaucaguguucuuggggg

SEQ ID NO: 109 - RNA, 81 nt, Homo sapiens
cuucuggaagcugguuucacaugguggcuuagauuuuuccaucuuuguaucuagcaccauuugaaaucaguguuuuaggag

SEQ ID NO: 110 - RNA, 88 nt, Homo sapiens
aucucuuacacaggcugaccgauuucuccugguguucagagucuguuuuugucuagcaccauuugaaaucgguuaugauguaggggga

SEQ ID NO: 111 - RNA, 76 nt, Homo sapiens
guacuugggcaguaguguagagauugguuugccuguuaaugaauucaaacuaaucucuacacugcugcccaagagc

SEQ ID NO: 112 - RNA, 82 nt, Homo sapiens
ccacgugccauguguacacacgugccaggcgcugucuugagacauucgcgcagugcacggcacuggggacacguggcacugg

SEQ ID NO: 113 - RNA, 70 nt, Homo sapiens
ggagauauugcacauuacuaaguugcauguugucacggccucaaugcaauuuagugugugugauauuuuc

SEQ ID NO: 114 - RNA, 95 nt, Homo sapiens
cucaucugucuguugggcuggaggcagggccuuugugaaggcggguggugcucagaucgccucugggcccuuccuccagccccgaggcggauuca

SEQ ID NO: 115 - RNA, 94 nt, Homo sapiens
cuuuggcgaucacugccucucugggccugugucuuaggcucugcaagaucaaccgagcaaagcacacggccugcagagaggcagcgcucugccc

SEQ ID NO: 116 - RNA, 75 nt, Homo sapiens
uguugucggguggaucacgaugcaauuuugaugaguaucauaggagaaaaauugcacgguauccaucuguaaacc

SEQ ID NO: 117 - RNA, 99 nt, Homo sapiens
ucuacaagcagauacaaggaugcccuuguacacaacacacgugcugcuuguauagacaugaguguugucuacgagggcauccuugugucugugugugug

SEQ ID NO: 118 - RNA, 95 nt, Homo sapiens
uguguuuuccucaacgcucacaguuacacuucuuacucucaauccauucauauugaaaaugaugaguagugacugaugaagcacaaaucagccaa

SEQ ID NO: 119 - RNA, 68 nt, Homo sapiens
ccauuacuguugcuaauaugcaacucuguugaauauaaauuggaauugcacuuuagcaauggugaugg

SEQ ID NO: 120 - RNA, 67 nt, Homo sapiens
gugggccucaaauguggagcacuauucugauguccaaguggaaagugcugcgacauuugagcgucac

SEQ ID NO: 121 - RNA, 69 nt, Homo sapiens
gggauacucaaaaugggggcgcuuuccuuuuugucuguacugggaagugcuucgauuuugggguguccc

SEQ ID NO: 122 - RNA, 71 nt, Homo sapiens
ugccaaugccuaucacauaucugccuguccuaugacaaacauggcagguagauauuugauaggcauuggca

SEQ ID NO: 123 - RNA, 54 nt, Homo sapiens
gaaagcugcaggugcugauguuggggggacauucagacuaccugcagcagagcc

SEQ ID NO: 124 - RNA, 58 nt, Homo sapiens
ugcucuguggagcugaggagcagauucucucucucuccucccggcuucaccuccugag

SEQ ID NO: 125 - RNA, 75 nt, Homo sapiens
gagcgcacagagguagguguggaagaaagugaaacacuauuuuagguuuuaguuacacucugcuguggugugcug

SEQ ID NO: 126 - RNA, 70 nt, Homo sapiens
cauguguccccuggcacgcuauuugagguuuacuauggaaccucaaguagucugaccaggggacacauga

SEQ ID NO: 127 - RNA, 76 nt, Homo sapiens
caggagagaaaguacugcccagaagcuaaaguguagaucaaacgcauaauggcugagguaguaguuucuugaacuu

SEQ ID NO: 128 - RNA, 65 nt, Homo sapiens
gcugcccuucacucagagcaucuacacccacuaccggugaguguggauccuggaggaaucguggc

SEQ ID NO: 129 - RNA, 89 nt, Homo sapiens
acgcaaaaugaacugaaccaggagugagcuucguguacauuaucuauuagaaaaugaaguaccuucugguucagcuagucccugugcgu

SEQ ID NO: 130 - RNA, 85 nt, Homo sapiens
ucaggcaaagggauauuuacagauacuuuuuaaaauuuguuugaguugaggcagauuaaauaucuguauucuccuuugccugcag

SEQ ID NO: 131 - RNA, 58 nt, Homo sapiens
gaguuauggggucaucuauccuucccuuggaaaaugaucugagaugacacuguagcuc

SEQ ID NO: 132 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 133 - RNA, 85 nt, Homo sapiens
cucaggcugugacccuccagagggaaguacuuucuguugucugagagaaaagaaagugcuucccuuuggacuguuucgguuugag

SEQ ID NO: 134 - RNA, 61 nt, Homo sapiens
cccucuacagggaagcgcuuucuguugucugaaagaaaagaaagugcuuccuuuuagaggg

SEQ ID NO: 135 - RNA, 87 nt, Homo sapiens
ucucaggcugucguccucuagagggaagcacuuucuguugucugaaagaaaagaaagugcuuccuuuuagaggguuaccguuugaga

SEQ ID NO: 136 - RNA, 87 nt, Homo sapiens
ucucaagcugugagucuacaaagggaagcccuuucuguugucuaaaagaaaagaaagugcuucucuuugguggguuacgguuugaga

SEQ ID NO: 137 - RNA, 87 nt, Homo sapiens; identical to SEQ ID NO: 136

SEQ ID NO: 138 - RNA, 87 nt, Homo sapiens
ucuccugcugugacccucaagauggaagcaguuucuguugucugaaaggaaagaaagugcuuccuuuuugaggguuacuguuugaga

SEQ ID NO: 139 - RNA, 87 nt, Homo sapiens
ucucaugcugugacccuacaaagggaagcacuuucucuuguccaaaggaaaagaaggcgcuucccuuuggaguguuacgguuugaga

SEQ ID NO: 140 - RNA, 77 nt, Homo sapiens
gaguugggagguucccucuccaaaugugucuugaucccccaccccaagacacauuuggagagggacccucccaacuc

SEQ ID NO: 141 - RNA, 78 nt, Homo sapiens
cugaaauagguugccugugagguguucacuuucuauaugaugaauauuauacagucaaccucuuuccgauaucgaauc

SEQ ID NO: 142 - RNA, 109 nt, Homo sapiens
gcuuuuauauuguagguuuuugcucaugcaccaugguugucugagcaugcagcaugcuugucugcucauaccccaugguuucugagcaggaaccuucauugucuacugc

SEQ ID NO: 143 - RNA, 78 nt, Homo sapiens
cuuucuacacagguugggaucgguugcaaugcuguguuucuguaugguauugcacuugucccggccuguugaguuugg

SEQ ID NO: 144 - RNA, 75 nt, Homo sapiens
ucaucccuggguggggauuuguugcauuacuuguguucuauauaaaguauugcacuugucccggccuguggaaga

SEQ ID NO: 145 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 146 - RNA, 80 nt, Homo sapiens
cugggggcuccaaagugcuguucgugcagguagugugauuacccaaccuacugcugagcuagcacuucccgagcccccgg

SEQ ID NO: 147 - RNA, 119 nt, Homo sapiens
aggauucugcucaugccagggugagguaguaaguuguauuguugugggguagggauauuaggccccaauuagaagauaacuauacaacuuacuacuuucccugguguguggcauauuca

SEQ ID NO: 148 - RNA, 21 nt, Homo sapiens
aauauaacacagauggccugu

SEQ ID NO: 149 - RNA, 22 nt, Homo sapiens
uauaaaaugagggcaguaagac

SEQ ID NO: 150 - RNA, 22 nt, Homo sapiens
ucagugcacuacagaacuuugu

SEQ ID NO: 151 - RNA, 22 nt, Homo sapiens
ucagugcaucacagaacuuugu

SEQ ID NO: 152 - RNA, 21 nt, Homo sapiens
ucagugcaugacagaacuugg

SEQ ID NO: 153 - RNA, 22 nt, Homo sapiens
uaaauagaguaggcaaaggaca

SEQ ID NO: 154 - RNA, 22 nt, Homo sapiens
aaacaaacauggugcacuucuu

SEQ ID NO: 155 - RNA, 21 nt, Homo sapiens
ucugaauuguaagaguuguua

SEQ ID NO: 156 - RNA, 80 nt, Homo sapiens
gguaccugagaagagguugucugugaugaguucgcuuuuauuaaugacgaauauaacacagauggccuguuuucaguacc

SEQ ID NO: 157 - RNA, 73 nt, Homo sapiens
uuccucaucuauaaaaugagggcaguaagaccuuccuuccuugucuuacuacccccauuuuauagaugaggaa

SEQ ID NO: 158 - RNA, 68 nt, Homo sapiens
gaggcaaaguucugagacacuccgacucugaguaugauagaagucagugcacuacagaacuuugucuc

SEQ ID NO: 159 - RNA, 99 nt, Homo sapiens
caagcacgauuagcauuugaggugaaguucuguuauacacucaggcuguggcucucugaaagucagugcaucacagaacuuugucucgaaagcuuucua

SEQ ID NO: 160 - RNA, 87 nt, Homo sapiens
ugucccccccggcccagguucugugauacacuccgacucgggcucuggagcagucagugcaugacagaacuugggcccggaaggacc

SEQ ID NO: 161 - RNA, 77 nt, Homo sapiens
aaaugguuauguccuuugccuauucuauuuaagacacccuguaccuuaaauagaguaggcaaaggacagaaacauuu

SEQ ID NO: 162 - RNA, 82 nt, Homo sapiens
ugguaccugaaaagaaguugcccauguuauuuucgcuuuauaugugacgaaacaaacauggugcacuucuuuuucgguauca

SEQ ID NO: 163 - RNA, 66 nt, Homo sapiens
uauaagaacucuugcagucuuagauguuauaaaaauauauaucugaauuguaagaguuguuagcac

SEQ ID NO: 164 - RNA, 22 nt, Homo sapiens
uauugcacuugucccggccugu

SEQ ID NO: 165 - RNA, 22 nt, Homo sapiens
uauugcacuugucccggccugu

SEQ ID NO: 166 - RNA, 22 nt, Homo sapiens
uagcuuaucagacugauguuga

SEQ ID NO: 167 - RNA, 22 nt, Homo sapiens
uucaaguaauccaggauaggcu

SEQ ID NO: 168 - RNA, 22 nt, Homo sapiens
uucaaguaauccaggauaggcu

SEQ ID NO: 169 - RNA, 23 nt, Homo sapiens
uaaggugcaucuagugcagauag

SEQ ID NO: 170 - RNA, 20 nt, Homo sapiens
uaaggcacgcggugaaugcc

SEQ ID NO: 171 - RNA, 20 nt, Homo sapiens
uaaggcacgcggugaaugcc

SEQ ID NO: 172 - RNA, 20 nt, Homo sapiens
uaaggcacgcggugaaugcc

SEQ ID NO: 173 - RNA, 22 nt, Homo sapiens
aacccguagauccgaucuugug

SEQ ID NO: 174 - RNA, 23 nt, Homo sapiens
uguaaacauccuacacucucagc

SEQ ID NO: 175 - RNA, 23 nt, Homo sapiens
cagugcaauaguauugucaaagc

SEQ ID NO: 176 - RNA, 23 nt, Homo sapiens
guccaguuuucccaggaaucccu

SEQ ID NO: 177 - RNA, 22 nt, Homo sapiens
ggugcagugcugcaucucuggu

SEQ ID NO: 178 - RNA, 23 nt, Homo sapiens
gaagugcuucgauuuuggggugu

SEQ ID NO: 179 - RNA, 23 nt, Homo sapiens
caaagugcucauagugcagguag

SEQ ID NO: 180 - RNA, 22 nt, Homo sapiens
uagcaccauuugaaaucgguua

SEQ ID NO: 181 - RNA, 23 nt, Homo sapiens
uagcaccauuugaaaucaguguu

SEQ ID NO: 182 - RNA, 22 nt, Homo sapiens
ugagguaguaguuuguacaguu

SEQ ID NO: 183 - RNA, 22 nt, Homo sapiens
ugagguaguagguuguauaguu

SEQ ID NO: 184 - RNA, 22 nt, Homo sapiens
ugagguaguagguugugugguu

SEQ ID NO: 185 - RNA, 22 nt, Homo sapiens
ugagguaguaaguuguauuguu

SEQ ID NO: 186 - RNA, 22 nt, Homo sapiens
cuuucagucggauguuugcagc

SEQ ID NO: 187 - RNA, 23 nt, Homo sapiens
caaagugcuuacagugcagguag

SEQ ID NO: 188 - RNA, 22 nt, Homo sapiens
uggaauguaaagaaguauguau

SEQ ID NO: 189 - RNA, 22 nt, Homo sapiens
uggaauguaaagaaguauguau

SEQ ID NO: 190 - RNA, 21 nt, Homo sapiens
cugaccuaugaauugacagcc

SEQ ID NO: 191 - RNA, 23 nt, Homo sapiens
uuaaugcuaaucgugauaggggu

SEQ ID NO: 192 - RNA, 23 nt, Homo sapiens
uucucgaggaaagaagcacuuuc

SEQ ID NO: 193 - RNA, 18 nt, Homo sapiens
ugcuuccuuucagagggu

SEQ ID NO: 194 - RNA, 21 nt, Homo sapiens
aggcaagaugcuggcauagcu

SEQ ID NO: 195 - RNA, 23 nt, Homo sapiens
aacauucaacgcugucggugagu

SEQ ID NO: 196 - RNA, 23 nt, Homo sapiens
aacauucaacgcugucggugagu

SEQ ID NO: 197 - RNA, 23 nt, Homo sapiens
aacauucauugcugucggugggu

SEQ ID NO: 198 - RNA, 23 nt, Homo sapiens
aacauucauugcugucggugggu

SEQ ID NO: 199 - RNA, 22 nt, Homo sapiens
aacauucaaccugucggugagu

SEQ ID NO: 200 - RNA, 23 nt, Homo sapiens
aggcaguguaguuagcugauugc

SEQ ID NO: 201 - RNA, 23 nt, Homo sapiens
uaggcagugucauuagcugauug

SEQ ID NO: 202 - RNA, 23 nt, Homo sapiens
agcagcauuguacagggcuauga

SEQ ID NO: 203 - RNA, 23 nt, Homo sapiens
agcagcauuguacagggcuauga

SEQ ID NO: 204 - RNA, 22 nt, Homo sapiens
cugugcgugugacagcggcuga

SEQ ID NO: 205 - RNA, 22 nt, Homo sapiens
uagcagcacguaaauauuggcg

SEQ ID NO: 206 - RNA, 22 nt, Homo sapiens
uagcagcacguaaauauuggcg

SEQ ID NO: 207 - RNA, 22 nt, Homo sapiens
uguaaacauccucgacuggaag

SEQ ID NO: 208 - RNA, 21 nt, Homo sapiens
aggcaagaugcuggcauagcu

SEQ ID NO: 209 - RNA, 21 nt, Homo sapiens
agcuacaucuggcuacugggu

SEQ ID NO: 210 - RNA, 23 nt, Homo sapiens
caaagugcuuacagugcagguag

SEQ ID NO: 211 - RNA, 22 nt, Homo sapiens
acugcagugaaggcacuuguag

SEQ ID NO: 212 - RNA, 22 nt, Homo sapiens
uaauacugccugguaaugauga

SEQ ID NO: 213 - RNA, 23 nt, Homo sapiens
uaauacugccggguaaugaugga

SEQ ID NO: 214 - RNA, 21 nt, Homo sapiens
ucacagugaaccggucucuuu

SEQ ID NO: 215 - RNA, 23 nt, Homo sapiens
uagcagcgggaacaguucugcag

SEQ ID NO: 216 - RNA, 22 nt, Homo sapiens
cagcagcaauucauguuuugaa

SEQ ID NO: 217 - RNA, 21 nt, Homo sapiens
uagcagcacagaaauauuggc

SEQ ID NO: 218 - RNA, 22 nt, Homo sapiens
aggcauugacuucucacuagcu

SEQ ID NO: 219 - RNA, 22 nt, Homo sapiens
gugaaauguuuaggaccacuag

SEQ ID NO: 220 - RNA, 22 nt, Homo sapiens
acaguagucugcacauugguua

SEQ ID NO: 221 - RNA, 23 nt, Homo sapiens
cccaguguucagacuaccuguuc

SEQ ID NO: 222 - RNA, 22 nt, Homo sapiens
acaguagucugcacauugguua

SEQ ID NO: 223 - RNA, 23 nt, Homo sapiens
caaagugcuguucgugcagguag

SEQ ID NO: 224 - RNA, 22 nt, Homo sapiens
ugagguaguaaguuguauuguu

SEQ ID NO: 225 - RNA, 24 nt, Homo sapiens
ucccugagacccuuuaaccuguga

SEQ ID NO: 226 - RNA, 22 nt, Homo sapiens
uuugguccccuucaaccagcug

SEQ ID NO: 227 - RNA, 22 nt, Homo sapiens
uuugguccccuucaaccagcua

SEQ ID NO: 228 - RNA, 22 nt, Homo sapiens
ucguaccgugaguaauaaugcg

SEQ ID NO: 229 - RNA, 22 nt, Homo sapiens
uguaacagcaacuccaugugga

SEQ ID NO: 230 - RNA, 23 nt, Homo sapiens
ugucugcccgcaugccugccucu

SEQ ID NO: 231 - RNA, 22 nt, Homo sapiens
uagcagcacaucaugguuuaca

SEQ ID NO: 232 - RNA, 22 nt, Homo sapiens
uccagcaucagugauuuuguug

SEQ ID NO: 233 - RNA, 22 nt, Homo sapiens
uccuucauuccaccggagucug

SEQ ID NO: 234 - RNA, 22 nt, Homo sapiens
ucaccagcccuguguucccuag

SEQ ID NO: 235 - RNA, 23 nt, Homo sapiens
aaggagcuuacaaucuagcuggg

SEQ ID NO: 236 - RNA, 22 nt, Homo sapiens
uggcaguguauuguuagcuggu

SEQ ID NO: 237 - RNA, 22 nt, Homo sapiens
acuggacuuagggucagaaggc

SEQ ID NO: 238 - RNA, 22 nt, Homo sapiens
uuauaaagcaaugagacugauu

SEQ ID NO: 239 - RNA, 23 nt, Homo sapiens
uaaaucccauggugccuucuccu

SEQ ID NO: 240 - RNA, 22 nt, Homo sapiens
aaaaugguucccuuuagagugu

SEQ ID NO: 241 - RNA, 22 nt, Homo sapiens
aggcggggcgccgcgggaccgc

SEQ ID NO: 242 - RNA, 22 nt, Homo sapiens
cagugcaauguuaaaagggcau

SEQ ID NO: 243 - RNA, 22 nt, Homo sapiens
cagugcaaugaugaaagggcau

SEQ ID NO: 244 - RNA, 22 nt, Homo sapiens
ucuucucuguuuuggccaugug

SEQ ID NO: 245 - RNA, 20 nt, Homo sapiens
guccgcucggcgguggccca

SEQ ID NO: 246 - RNA, 23 nt, Homo sapiens
aucgcugcgguugcgagcgcugu

SEQ ID NO: 247 - RNA, 22 nt, Homo sapiens
uggugggccgcagaacaugugc

SEQ ID NO: 248 - RNA, 22 nt, Homo sapiens
uucccuuugucauccuaugccu

SEQ ID NO: 249 - RNA, 21 nt, Homo sapiens
caagucacuagugguuccguu

SEQ ID NO: 250 - RNA, 22 nt, Homo sapiens
agucauuggaggguuugagcag

SEQ ID NO: 251 - RNA, 22 nt, Homo sapiens
uggagugugacaaugguguuug

SEQ ID NO: 252 - RNA, 22 nt, Homo sapiens
uaugugggaugguaaaccgcuu

SEQ ID NO: 253 - RNA, 22 nt, Homo sapiens
ugguuuaccgucccacauacau

SEQ ID NO: 254 - RNA, 22 nt, Homo sapiens
aacccguagauccgaacuugug

SEQ ID NO: 255 - RNA, 23 nt, Homo sapiens
agcugguguugugaaucaggccg

SEQ ID NO: 256 - RNA, 22 nt, Homo sapiens
cagugguuuuacccuaugguag

SEQ ID NO: 257 - RNA, 22 nt, Homo sapiens
uuuguucguucggcucgcguga

SEQ ID NO: 258 - RNA, 23 nt, Homo sapiens
uacugcaucaggaacugauugga

SEQ ID NO: 259 - RNA, 23 nt, Homo sapiens
aaagugcugcgacauuugagcgu

SEQ ID NO: 260 - RNA, 23 nt, Homo sapiens
uuuggcacuagcacauuuuugcu

SEQ ID NO: 261 - RNA, 22 nt, Homo sapiens
ucggauccgucugagcuuggcu

SEQ ID NO: 262 - RNA, 23 nt, Homo sapiens
uauggcuuuucauuccuauguga

SEQ ID NO: 263 - RNA, 21 nt, Homo sapiens
uacaguacugugauaacugaa

SEQ ID NO: 264 - RNA, 20 nt, Homo sapiens
ccucugggcccuuccuccag

SEQ ID NO: 265 - RNA, 23 nt, Homo sapiens
cgcauccccuagggcauuggugu

SEQ ID NO: 266 - RNA, 20 nt, Homo sapiens
acugccccaggugcugcugg

SEQ ID NO: 267 - RNA, 23 nt, Homo sapiens
ucaagagcaauaacgaaaaaugu

SEQ ID NO: 268 - RNA, 22 nt, Homo sapiens
uaacacugucugguaaagaugg

SEQ ID NO: 269 - RNA, 78 nt, Homo sapiens; identical to SEQ ID NO: 143

SEQ ID NO: 270 - RNA, 75 nt, Homo sapiens; identical to SEQ ID NO: 144

SEQ ID NO: 271 - RNA, 72 nt, Homo sapiens
ugucggguagcuuaucagacugauguugacuguugaaucucauggcaacaccagucgaugggcugucugaca

SEQ ID NO: 272 - RNA, 77 nt, Homo sapiens; identical to SEQ ID NO: 104

SEQ ID NO: 273 - RNA, 84 nt, Homo sapiens; identical to SEQ ID NO: 105

SEQ ID NO: 274 - RNA, 71 nt, Homo sapiens
uguucuaaggugcaucuagugcagauagugaaguagauuagcaucuacugcccuaagugcuccuucuggca

SEQ ID NO: 275 - RNA, 87 nt, Homo sapiens
ugagggccccucugcguguucacagcggaccuugauuuaaugucuauacaauuaaggcacgcggugaaugccaagagaggcgccucc

SEQ ID NO: 276 - RNA, 85 nt, Homo sapiens
aggccucucucuccguguucacagcggaccuugauuuaaauguccauacaauuaaggcacgcggugaaugccaagaauggggcug

SEQ ID NO: 277 - RNA, 109 nt, Homo sapiens
aucaagauuagaggcucugcucuccguguucacagcggaccuugauuuaaugucauacaauuaaggcacgcggugaaugccaagagcggagccuacggcugcacuugaa

SEQ ID NO: 278 - RNA, 81 nt, Homo sapiens
cccauuggcauaaacccguagauccgaucuuguggugaaguggaccgcacaagcucgcuucuaugggucugugucagugug

SEQ ID NO: 279 - RNA, 89 nt, Homo sapiens
accaugcuguaguguguguaaacauccuacacucucagcugugagcucaagguggcugggagaggguuguuuacuccuucugccaugga

SEQ ID NO: 280 - RNA, 72 nt, Homo sapiens
agauacuguaaacauccuacacucucagcuguggaaaguaagaaagcugggagaaggcuguuuacucuuucu

SEQ ID NO: 281 - RNA, 86 nt, Homo sapiens
acugcuaacgaaugcucugacuuuauugcacuacuguacuuuacagcuagcagugcaauaguauugucaaagcaucugaaagcagg

SEQ ID NO: 282 - RNA, 88 nt, Homo sapiens; identical to SEQ ID NO: 89

SEQ ID NO: 283 - RNA, 106 nt, Homo sapiens; identical to SEQ ID NO: 87

SEQ ID NO: 284 - RNA, 69 nt, Homo sapiens; identical to SEQ ID NO: 121

SEQ ID NO: 285 - RNA, 69 nt, Homo sapiens; identical to SEQ ID NO: 101

SEQ ID NO: 286 - RNA, 88 nt, Homo sapiens; identical to SEQ ID NO: 110

SEQ ID NO: 287 - RNA, 81 nt, Homo sapiens; identical to SEQ ID NO: 108

SEQ ID NO: 288 - RNA, 81 nt, Homo sapiens; identical to SEQ ID NO: 109

SEQ ID NO: 289 - RNA, 84 nt, Homo sapiens; identical to SEQ ID NO: 81

SEQ ID NO: 290 - RNA, 80 nt, Homo sapiens; identical to SEQ ID NO: 73

SEQ ID NO: 291 - RNA, 72 nt, Homo sapiens; identical to SEQ ID NO: 74

SEQ ID NO: 292 - RNA, 74 nt, Homo sapiens; identical to SEQ ID NO: 75

SEQ ID NO: 293 - RNA, 83 nt, Homo sapiens; identical to SEQ ID NO: 76

SEQ ID NO: 294 - RNA, 119 nt, Homo sapiens; identical to SEQ ID NO: 147

SEQ ID NO: 295 - RNA, 71 nt, Homo sapiens
gcgacuguaaacauccucgacuggaagcugugaagccacagaugggcuuucagucggauguuugcagcugc

SEQ ID NO: 296 - RNA, 84 nt, Homo sapiens; identical to SEQ ID NO: 90

SEQ ID NO: 297 - RNA, 71 nt, Homo sapiens
ugggaaacauacuucuuuauaugcccauauggaccugcuaagcuauggaauguaaagaaguauguaucuca

SEQ ID NO: 298 - RNA, 85 nt, Homo sapiens
accuacucagaguacauacuucuuuauguacccauaugaacauacaaugcuauggaauguaaagaaguauguauuuuugguaggc

SEQ ID NO: 299 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 300 - RNA, 65 nt, Homo sapiens
cuguuaaugcuaaucgugauagggguuuuugccuccaacugacuccuacauauuagcauuaacag

SEQ ID NO: 301 - RNA, 90 nt, Homo sapiens
ucucaggcugugaccuucucgaggaaagaagcacuuucuguugucugaaagaaaagaaagugcuuccuuucagaggguuacgguuugaga

SEQ ID NO: 302 - RNA, 90 nt, Homo sapiens
ucucagguugugaccuucucgaggaaagaagcacuuucuguugucugaaagaaaagaaagugcuuccuuucagaggguuacgguuugaga

SEQ ID NO: 303 - RNA, 71 nt, Homo sapiens
ggagaggaggcaagaugcuggcauagcuguugaacugggaaccugcuaugccaacauauugccaucuuucc

SEQ ID NO: 304 - RNA, 110 nt, Homo sapiens; identical to SEQ ID NO: 91

SEQ ID NO: 305 - RNA, 110 nt, Homo sapiens; identical to SEQ ID NO: 92

SEQ ID NO: 306 - RNA, 110 nt, Homo sapiens; identical to SEQ ID NO: 93

SEQ ID NO: 307 - RNA, 89 nt, Homo sapiens; identical to SEQ ID NO: 94

SEQ ID NO: 308 - RNA, 110 nt, Homo sapiens; identical to SEQ ID NO: 95

SEQ ID NO: 309 - RNA, 77 nt, Homo sapiens
agucuaguuacuaggcaguguaguuagcugauugcuaauaguaccaaucacuaaccacacggccagguaaaaagauu

SEQ ID NO: 310 - RNA, 84 nt, Homo sapiens
gugcucgguuuguaggcagugucauuagcugauuguacuguggugguuacaaucacuaacuccacugccaucaaaacaaggcac

SEQ ID NO: 311 - RNA, 78 nt, Homo sapiens
uacugcccucggcuucuuuacagugcugccuuguugcauauggaucaagcagcauuguacagggcuaugaaggcauug

SEQ ID NO: 312 - RNA, 78 nt, Homo sapiens
uugugcuuucagcuucuuuacagugcugccuuguagcauucaggucaagcagcauuguacagggcuaugaaagaacca

SEQ ID NO: 313 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 314 - RNA, 89 nt, Homo sapiens
gucagcagugccuuagcagcacguaaauauuggcguuaagauucuaaaauuaucuccaguauuaacugugcugcugaaguaagguugac

SEQ ID NO: 315 - RNA, 81 nt, Homo sapiens
guuccacucuagcagcacguaaauauuggcguagugaaauauauauuaaacaccaauauuacugugcugcuuuagugugac

SEQ ID NO: 316 - RNA, 71 nt, Homo sapiens; identical to SEQ ID NO: 295

SEQ ID NO: 317 - RNA, 71 nt, Homo sapiens; identical to SEQ ID NO: 303

SEQ ID NO: 318 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 319 - RNA, 84 nt, Homo sapiens; identical to SEQ ID NO: 90

SEQ ID NO: 320 - RNA, 84 nt, Homo sapiens; identical to SEQ ID NO: 90

SEQ ID NO: 321 - RNA, 95 nt, Homo sapiens
ccagcucgggcagccguggccaucuuacugggcagcauuggauggagucaggucucuaauacugccugguaaugaugacggcggagcccugcacg

SEQ ID NO: 322 - RNA, 68 nt, Homo sapiens
cccucgucuuacccagcaguguuugggugcgguugggagucucuaauacugccggguaaugauggagg

SEQ ID NO: 323 - RNA, 82 nt, Homo sapiens
ugagcuguuggauucggggccguagcacugucugagagguuuacauuucucacagugaaccggucucuuuuucagcugcuuc

SEQ ID NO: 324 - RNA, 84 nt, Homo sapiens
ugugcagugggaaggggggccgauacacuguacgagagugaguagcaggucucacagugaaccggucucuuucccuacuguguc

SEQ ID NO: 325 - RNA, 71 nt, Homo sapiens
ugcccuagcagcgggaacaguucugcagugagcgaucggugcucugggguauuguuuccgcugccagggua

SEQ ID NO: 326 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 327 - RNA, 87 nt, Homo sapiens
agcuucccuggcucuagcagcacagaaauauuggcacagggaagcgagucugccaauauuggcugugcugcuccaggcaggguggug

SEQ ID NO: 328 - RNA, 119 nt, Homo sapiens
agucagccuguugaagcuuugaagcuuugaugccaggcauugacuucucacuagcugugaaaguccuagcuaaagagaagucaaugcaugacaucuuguuucaauagauggcuguuuca

SEQ ID NO: 329 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 330 - RNA, 71 nt, Homo sapiens; identical to SEQ ID NO: 97

SEQ ID NO: 331 - RNA, 71 nt, Homo sapiens; identical to SEQ ID NO: 97

SEQ ID NO: 332 - RNA, 110 nt, Homo sapiens; identical to SEQ ID NO: 98

SEQ ID NO: 333 - RNA, 80 nt, Homo sapiens; identical to SEQ ID NO: 146

SEQ ID NO: 334 - RNA, 119 nt, Homo sapiens; identical to SEQ ID NO: 147

SEQ ID NO: 335 - RNA, 86 nt, Homo sapiens
ugccagucucuaggucccugagacccuuuaaccugugaggacauccagggucacaggugagguucuugggagccuggcgucuggcc

SEQ ID NO: 336 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 337 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 338 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 339 - RNA, 85 nt, Homo sapiens
cgcuggcgacgggacauuauuacuuuugguacgcgcugugacacuucaaacucguaccgugaguaauaaugcgccguccacggca

SEQ ID NO: 340 - RNA, 85 nt, Homo sapiens
augguguuaucaaguguaacagcaacuccauguggacuguguaccaauuuccaguggagaugcuguuacuuuugaugguuaccaa

SEQ ID NO: 341 - RNA, 85 nt, Homo sapiens
ugguucccgcccccuguaacagcaacuccauguggaagugcccacugguuccaguggggcugcuguuaucuggggcgagggccag

SEQ ID NO: 342 - RNA, 95 nt, Homo sapiens
ggucucuguguugggcgucugucugcccgcaugccugccucucuguugcucugaaggaggcaggggcugggccugcagcugccugggcagagcgg

SEQ ID NO: 343 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 344 - RNA, 67 nt, Homo sapiens
ucuccaacaauauccuggugcugagugaugacucaggcgacuccagcaucagugauuuuguugaaga

SEQ ID NO: 345 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 346 - RNA, 75 nt, Homo sapiens
gugagggcaugcaggccuggauggggcagcugggaugguccaaaaggguggccucaccagcccuguguucccuag

SEQ ID NO: 347 - RNA, 88 nt, Homo sapiens
aacugcccucaaggagcuuacaaucuagcuggggguaaaugacuugcacaugaacacaacuagacugugagcuucuagagggcaggga

SEQ ID NO: 348 - RNA, 91 nt, Homo sapiens
cugugugugaugagcuggcaguguauuguuagcugguugaauaugugaauggcaucggcuaacaugcaacugcugucuuauugcauauaca

SEQ ID NO: 349 - RNA, 90 nt, Homo sapiens
gagagaagcacuggacuuagggucagaaggccugagucucucugcugcagaugggcucucugucccugagccaagcuuuguccucccugg

SEQ ID NO: 350 - RNA, 95 nt, Homo sapiens
uuguaccuggugugauuauaaagcaaugagacugauugucauaugucguuugugggauccgucucaguuacuuuauagccauaccugguaucuua

SEQ ID NO: 351 - RNA, 83 nt, Homo sapiens
gcccuagcuugguucuaaaucccauggugccuucuccuugggaaaaacagagaaggcacuaugagauuuagaaucaaguuagg

SEQ ID NO: 352 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 353 - RNA, 93 nt, Homo sapiens
ccuuccggcgucccaggcggggcgccgcgggaccgcccucgugucuguggcggugggaucccgcggccguguuuuccugguggcccggccaug

SEQ ID NO: 354 - RNA, 89 nt, Homo sapiens
ugcugcuggccagagcucuuuucacauugugcuacugucugcaccugucacuagcagugcaauguuaaaagggcauuggccguguagug

SEQ ID NO: 355 - RNA, 82 nt, Homo sapiens
ggccugcccgacacucuuucccuguugcacuacuauaggccgcugggaagcagugcaaugaugaaagggcaucggucagguc

SEQ ID NO: 356 - RNA, 86 nt, Homo sapiens
auuaggagaguaucuucucuguuuuggccauguguguacucacagccccucacacauggccgaaacagagaaguuacuuuccuaau

SEQ ID NO: 357 - RNA, 95 nt, Homo sapiens
gucgaggccguggcccggaaguggucggggccgcugcgggcggaagggcgccugugcuucguccgcucggcgguggcccagccaggcccgcggga

SEQ ID NO: 358 - RNA, 98 nt, Homo sapiens
uggccgacggggcgcgcgcggccuggaggggcggggcggacgcagagccgcguuuagucuaucgcugcgguugcgagcgcuguagggagccugugcug

SEQ ID NO: 359 - RNA, 81 nt, Homo sapiens
ggguaaguggaaagauggugggccgcagaacaugugcugaguucgugccauaugucugcugaccaucaccuuuagaagccc

SEQ ID NO: 360 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 361 - RNA, 81 nt, Homo sapiens
gggcuuucaagucacuagugguuccguuuaguagaugauugugcauuguuucaaaauggugcccuagugacuacaaagccc

SEQ ID NO: 362 - RNA, 97 nt, Homo sapiens
uuagguaauuccuccacucaaaacccuucagugacuuccaugacaugaaauaggaagucauuggaggguuugagcagaggaaugaccuguuuuaaaa

SEQ ID NO: 363 - RNA, 85 nt, Homo sapiens
ccuuagcagagcuguggagugugacaaugguguuugugucuaaacuaucaaacgccauuaucacacuaaauagcuacugcuaggc

SEQ ID NO: 364 - RNA, 63 nt, Homo sapiens
aagaaaugguuuaccgucccacauacauuuugaauauguaugugggaugguaaaccgcuucuu

SEQ ID NO: 365 - RNA, 80 nt, Homo sapiens
ccuguugccacaaacccguagauccgaacuugugguauuaguccgcacaagcuuguaucuauagguaugugucuguuagg

SEQ ID NO: 366 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 367 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 368 - RNA, 64 nt, Homo sapiens
ccccgcgacgagccccucgcacaaaccggaccugagcguuuuguucguucggcucgcgugaggc

SEQ ID NO: 369 - RNA, 110 nt, Homo sapiens
aguauaauuauuacauaguuuuugaugucgcagauacugcaucaggaacugauuggauaagaaucagucaccaucaguuccuaaugcauugccuucagcaucuaaacaag

SEQ ID NO: 370 - RNA, 67 nt, Homo sapiens; identical to SEQ ID NO: 120

SEQ ID NO: 371 - RNA, 78 nt, Homo sapiens
uggccgauuuuggcacuagcacauuuuugcuugugucucuccgcucugagcaaucaugugcagugccaauaugggaaa

SEQ ID NO: 372 - RNA, 97 nt, Homo sapiens
ugugaucacugucuccagccugcugaagcucagagggcucugauucagaaagaucaucggauccgucugagcuuggcuggucggaagucucaucauc

SEQ ID NO: 373 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 374 - RNA, 75 nt, Homo sapiens
ugcccuggcucaguuaucacagugcugaugcugucuauucuaaagguacaguacugugauaacugaaggauggca

SEQ ID NO: 375 - RNA, 79 nt, Homo sapiens
acuguccuuuuucgguuaucaugguaccgaugcuguauaucugaaagguacaguacugugauaacugaagaaugguggu

SEQ ID NO: 376 - RNA, 95 nt, Homo sapiens; identical to SEQ ID NO: 114

SEQ ID NO: 377 - RNA, 83 nt, Homo sapiens
cugacuaugccuccccgcauccccuagggcauugguguaaagcuggagacccacugccccaggugcugcuggggguuguaguc

SEQ ID NO: 378 - RNA, 83 nt, Homo sapiens; identical to SEQ ID NO: 377

SEQ ID NO: 379 - RNA, 94 nt, Homo sapiens
uguuuugagcgggggucaagagcaauaacgaaaaauguuugucauaaaccguuuuucauuauugcuccugaccuccucucauuugcuauauuca

SEQ ID NO: 380 - RNA, 95 nt, Homo sapiens
cggccggcccuggguccaucuuccaguacaguguuggauggucuaauugugaagcuccuaacacugucugguaaagauggcucccggguggguuc

SEQ ID NO: 381 - RNA, 17 nt, Homo sapiens
gugggggagaggcuguc

SEQ ID NO: 382 - RNA, 22 nt, Homo sapiens
ugcaacgaaccugagccacuga

SEQ ID NO: 383 - RNA, 22 nt, Homo sapiens
uagguuauccguguugccuucg

SEQ ID NO: 384 - RNA, 21 nt, Homo sapiens
gugccagcugcagugggggag

SEQ ID NO: 385 - RNA, 20 nt, Homo sapiens
guccgcucggcgguggccca

SEQ ID NO: 386 - RNA, 23 nt, Homo sapiens
ccaguuaccgcuuccgcuaccgc

SEQ ID NO: 387 - RNA, 17 nt, Homo sapiens
acauugccagggaguuu

SEQ ID NO: 388 - RNA, 22 nt, Homo sapiens
uugcauagucacaaaagugauc

SEQ ID NO: 389 - RNA, 17 nt, Homo sapiens
uugucugcugaguuucc

SEQ ID NO: 390 - RNA, 23 nt, Homo sapiens
agguuacccgagcaacuuugcau

SEQ ID NO: 391 - RNA, 22 nt, Homo sapiens
ugggucuuugcgggcgagauga

SEQ ID NO: 392 - RNA, 19 nt, Homo sapiens
aagugugcagggcacuggu

SEQ ID NO: 393 - RNA, 22 nt, Homo sapiens
uaaugccccuaaaaauccuuau

SEQ ID NO: 394 - RNA, 23 nt, Homo sapiens
uaauccuugcuaccugggugaga

SEQ ID NO: 395 - RNA, 22 nt, Homo sapiens
aguggggaacccuuccaugagg

SEQ ID NO: 396 - RNA, 22 nt, Homo sapiens
acaguagucugcacauugguua

SEQ ID NO: 397 - RNA, 22 nt, Homo sapiens
acaguagucugcacauugguua

SEQ ID NO: 398 - RNA, 23 nt, Homo sapiens
cccaguguucagacuaccuguuc

SEQ ID NO: 399 - RNA, 23 nt, Homo sapiens
cccaguguucagacuaccuguuc

SEQ ID NO: 400 - RNA, 21 nt, Homo sapiens
auuugugcuuggcucugucac

SEQ ID NO: 401 - RNA, 23 nt, Homo sapiens
aaagugcugcgacauuugagcgu

SEQ ID NO: 402 - RNA, 23 nt, Homo sapiens
gaagugcuucgauuuuggggugu

SEQ ID NO: 403 - RNA, 22 nt, Homo sapiens
ucuucucuguuuuggccaugug

SEQ ID NO: 404 - RNA, 22 nt, Homo sapiens
uggguggucuggagauuugugc

SEQ ID NO: 405 - RNA, 23 nt, Homo sapiens
uaaggugcaucuagugcagauag

SEQ ID NO: 406 - RNA, 23 nt, Homo sapiens
gagggucuugggagggaugugac

SEQ ID NO: 407 - RNA, 21 nt, Homo sapiens
agaggauacccuuuguauguu

SEQ ID NO: 408 - RNA, 20 nt, Homo sapiens
uaaagagcccuguggagaca

SEQ ID NO: 409 - RNA, 22 nt, Homo sapiens
aacuggcccucaaagucccgcu

SEQ ID NO: 410 - RNA, 20 nt, Homo sapiens
cuuccucgucugucugcccc

SEQ ID NO: 411 - RNA, 21 nt, Homo sapiens
uuaauaucggacaaccauugu

SEQ ID NO: 412 - RNA, 22 nt, Homo sapiens
gccugcugggguggaaccuggu

SEQ ID NO: 413 - RNA, 22 nt, Homo sapiens
caaaaaccacaguuucuuuugc

SEQ ID NO: 414 - RNA, 80 nt, Homo sapiens
ccucugugagaaagggugugggggagaggcugucuugugucuguaaguaugccaaacuuauuuuccccaaggcagaggga

SEQ ID NO: 415 - RNA, 79 nt, Homo sapiens
ccuuaauccuugcaacgaaccugagccacugauucaguaaaauacucaguggcacauguuuguugugagggucaaaaga

SEQ ID NO: 416 - RNA, 84 nt, Homo sapiens
gugguacuugaagauagguuauccguguugccuucgcuuuauuugugacgaaucauacacgguugaccuauuuuucaguaccaa

SEQ ID NO: 417 - RNA, 83 nt, Homo sapiens
ccugcugcagaggugccagcugcagugggggaggcacugccagggcugcccacucugcuuagccagcaggugccaagaacagg

SEQ ID NO: 418 - RNA, 95 nt, Homo sapiens; identical to SEQ ID NO: 357

SEQ ID NO: 419 - RNA, 91 nt, Homo sapiens
ggcgggggcgcgggcggcaguggcgggagcggccccucggccauccuccgucugcccaguuaccgcuuccgcuaccgccgccgcucccgcu

SEQ ID NO: 420 - RNA, 65 nt, Homo sapiens
aaaaggcgagacauugccagggaguuuauuuuguagcucucuugauaaaauguuuuagcaaacac

SEQ ID NO: 421 - RNA, 90 nt, Homo sapiens
cucacagcugccagugucauuuuugugaucugcagcuaguauucucacuccaguugcauagucacaaaagugaucauuggcagguguggc

SEQ ID NO: 422 - RNA, 87 nt, Homo sapiens
agcgguggccagugucauuuuugugauguugcagcuaguaauaugagcccaguugcauagucacaaaagugaucauuggaaacugug

SEQ ID NO: 423 - RNA, 67 nt, Homo sapiens
auggagguggagagucaucagcagcacugagcaggcaguguugucugcugaguuuccacgucauuug

SEQ ID NO: 424 - RNA, 79 nt, Homo sapiens
ugguacucggggagagguuacccgagcaacuuugcaucuggacgacgaauguugcucggugaaccccuuuucgguauca

SEQ ID NO: 425 - RNA, 88 nt, Homo sapiens
cgaggaugggagcugagggcugggucuuugcgggcgagaugagggugucggaucaacuggccuacaaagucccaguucucggcccccg

SEQ ID NO: 426 - RNA, 94 nt, Homo sapiens
aucacagacaccuccaagugugcagggcacuggugggggccggggcaggcccagcgaaagugcaggaccuggcacuuagucggaagugagggug

SEQ ID NO: 427 - RNA, 87 nt, Homo sapiens
accgcagggaaaaugagggacuuuugggggcagauguguuuccauuccacuaucauaaugccccuaaaaauccuuauugcucuugca

SEQ ID NO: 428 - RNA, 84 nt, Homo sapiens
gcucccccucucuaauccuugcuaccugggugagagugcugucugaaugcaaugcaccugggcaaggauucugagagcgagagc

SEQ ID NO: 429 - RNA, 84 nt, Homo sapiens
uugacuuagcuggguaguggggaacccuuccaugaggaguagaacacuccuuaugcaagauucccuucuaccuggcuggguugg

SEQ ID NO: 430 - RNA, 71 nt, Homo sapiens; identical to SEQ ID NO: 97

SEQ ID NO: 431 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 432 - RNA, 71 nt, Homo sapiens; identical to SEQ ID NO: 97

SEQ ID NO: 433 - RNA, 110 nt, Homo sapiens; identical to SEQ ID NO: 431

SEQ ID NO: 434 - RNA, 91 nt, Homo sapiens; identical to SEQ ID NO: 102

SEQ ID NO: 435 - RNA, 67 nt, Homo sapiens; identical to SEQ ID NO: 120

SEQ ID NO: 436 - RNA, 69 nt, Homo sapiens; identical to SEQ ID NO: 121

SEQ ID NO: 437 - RNA, 86 nt, Homo sapiens; identical to SEQ ID NO: 356

SEQ ID NO: 438 - RNA, 71 nt, Homo sapiens
agguuguucuggguggucuggagauuugugcagcuuguaccugcacaaaucuccggaccacuuagucuuua

SEQ ID NO: 439 - RNA, 71 nt, Homo sapiens; identical to SEQ ID NO: 274

SEQ ID NO: 440 - RNA, 97 nt, Homo sapiens
gggacuugucacugccugucuccucccucuccagcagcgacuggauucuggaguccaucuagagggucuugggagggaugugacuguugggaagccc

SEQ ID NO: 441 - RNA, 86 nt, Homo sapiens
uuugguacuugaagagaggauacccuuuguauguucacuugauuaauggcgaauauacagggggagacucuuauuugcguaucaaa

SEQ ID NO: 442 - RNA, 86 nt, Homo sapiens
uuugguacuuaaagagaggauacccuuuguauguucacuugauuaauggcgaauauacagggggagacucucauuugcguaucaaa

SEQ ID NO: 443 - RNA, 83 nt, Homo sapiens
ccccagcuagguaaagagcccuguggagacaccuggauucagagaacaugucuccacugagcacuugggccuugauggcggcu

SEQ ID NO: 444 - RNA, 83 nt, Homo sapiens
guggucucagaaucgggguuuugagggcgagaugaguuuauguuuuauccaacuggcccucaaagucccgcuuuuggggucau

SEQ ID NO: 445 - RNA, 83 nt, Homo sapiens
gugagugggagccccagugugugguuggggccauggcgggugggcagcccagccucugagccuuccucgucugucugccccag

SEQ ID NO: 446 - RNA, 79 nt, Homo sapiens
gugcuuaaagaauggcuguccguaguauggucucuauauuuaugaugauuaauaucggacaaccauuguuuuaguaucc

SEQ ID NO: 447 - RNA, 75 nt, Homo sapiens
agacagagaagccaggucacgucucugcaguuacacagcucacgagugccugcugggguggaaccuggucugucu

SEQ ID NO: 448 - RNA, 97 nt, Homo sapiens
aaacaaguuauauuagguuggugcaaaaguaauugugguuuuugccuguaaaaguaauggcaaaaaccacaguuucuuuugcaccagacuaauaaag

SEQ ID NO: 449 - RNA, 23 nt, Homo sapiens
caaagugcucauagugcagguag

SEQ ID NO: 450 - RNA, 23 nt, Homo sapiens
uaaggugcaucuagugcagauag

SEQ ID NO: 451 - RNA, 23 nt, Homo sapiens
caaagugcuuacagugcagguag

SEQ ID NO: 452 - RNA, 22 nt, Homo sapiens
uaacacugucugguaaagaugg

SEQ ID NO: 453 - RNA, 22 nt, Homo sapiens
acuuuaacauggaagugcuuuc

SEQ ID NO: 454 - RNA, 21 nt, Homo sapiens
uacaguacugugauaacugaa

SEQ ID NO: 455 - RNA, 21 nt, Homo sapiens
cauuauuacuuuugguacgcg

SEQ ID NO: 456 - RNA, 22 nt, Homo sapiens
ugagaacugaauuccauggguu

SEQ ID NO: 457 - RNA, 22 nt, Homo sapiens
ugagaacugaauuccauaggcu

SEQ ID NO: 458 - RNA, 22 nt, Homo sapiens
uucaaguaauccaggauaggcu

SEQ ID NO: 459 - RNA, 22 nt, Homo sapiens
uagcaccaucugaaaucgguua

SEQ ID NO: 460 - RNA, 22 nt, Homo sapiens
uaacagucuacagccauggucg

SEQ ID NO: 461 - RNA, 23 nt, Homo sapiens
ucuuugguuaucuagcuguauga

SEQ ID NO: 462 - RNA, 22 nt, Homo sapiens
ugagaacugaauuccauggguu

SEQ ID NO: 463 - RNA, 23 nt, Homo sapiens
uacccuguagaaccgaauuugug

SEQ ID NO: 464 - RNA, 21 nt, Homo sapiens
agcuacaucuggcuacugggu

SEQ ID NO: 465 - RNA, 22 nt, Homo sapiens
aacuggcccucaaagucccgcu

SEQ ID NO: 466 - RNA, 23 nt, Homo sapiens
agcuacauugucugcuggguuuc

SEQ ID NO: 467 - RNA, 23 nt, Homo sapiens
uauggcuuuuuauuccuauguga

SEQ ID NO: 468 - RNA, 23 nt, Homo sapiens
ucuggcuccgugucuucacuccc

SEQ ID NO: 469 - RNA, 23 nt, Homo sapiens
cccaguguucagacuaccuguuc

SEQ ID NO: 470 - RNA, 23 nt, Homo sapiens
acuuaaacguggauguacuugcu

SEQ ID NO: 471 - RNA, 22 nt, Homo sapiens
uuuaacauggggguaccugcug

SEQ ID NO: 472 - RNA, 23 nt, Homo sapiens
uaagugcuuccauguuugagugu

SEQ ID NO: 473 - RNA, 21 nt, Homo sapiens
aauaauacaugguugaucuuu

SEQ ID NO: 474 - RNA, 22 nt, Homo sapiens
gccugcugggguggaaccuggu

SEQ ID NO: 475 - RNA, 22 nt, Homo sapiens
ugagguaguagguuguauaguu

SEQ ID NO: 476 - RNA, 22 nt, Homo sapiens
ugagguaguagguugugugguu

SEQ ID NO: 477 - RNA, 23 nt, Homo sapiens
uacccuguagaaccgaauuugug

SEQ ID NO: 478 - RNA, 21 nt, Homo sapiens
aucacauugccagggauuucc

SEQ ID NO: 479 - RNA, 21 nt, Homo sapiens
aucacauugccagggauuacc

SEQ ID NO: 480 - RNA, 22 nt, Homo sapiens
uauugcacauuacuaaguugca

SEQ ID NO: 481 - RNA, 21 nt, Homo sapiens
gggagugcagggcaggguuuc

SEQ ID NO: 482 - RNA, 22 nt, Homo sapiens
agacccuggucugcacucuauc

SEQ ID NO: 483 - RNA, 83 nt, Homo sapiens
gcugcuguugggagacccuggucugcacucuaucuguauucuuacugaagggagugcagggcaggguuucccauacagagggc

SEQ ID NO: 484 - RNA, 23 nt, Homo sapiens
uaagugcuuccauguuuugguga

SEQ ID NO: 485 - RNA, 23 nt, Artificial
  Synthetic
auucacgaagguacaaaaccacu

SEQ ID NO: 486 - RNA, 23 nt, Homo sapiens
uaagugcuuccauguuuugguga

SEQ ID NO: 487 - RNA, 23 nt, Homo sapiens
acuuaaacguggauguacuugcu

SEQ ID NO: 488 - RNA, 69 nt, Homo sapiens
ccaccacuuaaacguggauguacuugcuuugaaacuaaagaaguaagugcuuccauguuuuggugaugg

SEQ ID NO: 489 - RNA, 23 nt, Homo sapiens
uaagugcuuccauguuuuaguag

SEQ ID NO: 490 - RNA, 22 nt, Homo sapiens
acuuuaacauggaagugcuuuc

SEQ ID NO: 491 - RNA, 73 nt, Homo sapiens
gcucccuucaacuuuaacauggaagugcuuucugugacuuuaaaaguaagugcuuccauguuuuaguaggagu

SEQ ID NO: 492 - RNA, 23 nt, Homo sapiens
uaagugcuuccauguuucagugg

SEQ ID NO: 493 - RNA, 22 nt, Homo sapiens
uuuaacauggggguaccugcug

SEQ ID NO: 494 - RNA, 68 nt, Homo sapiens
ccuuugcuuuaacauggggguaccugcugugugaaacaaaaguaagugcuuccauguuucaguggagg

SEQ ID NO: 495 - RNA, 23 nt, Homo sapiens
uaagugcuuccauguuugagugu

SEQ ID NO: 496 - RNA, 22 nt, Homo sapiens
acuuuaacauggaggcacuugc

SEQ ID NO: 497 - RNA, 68 nt, Homo sapiens
ccucuacuuuaacauggaggcacuugcugugacaugacaaaaauaagugcuuccauguuugagugugg

SEQ ID NO: 498 - RNA, 17 nt, Homo sapiens
uaagugcuuccaugcuu

SEQ ID NO: 499 - RNA, 72 nt, Homo sapiens
uuggguaagugcuuccaugcuucaguuuccuuacugguaagauggauguaguaauagcaccuaccuuauaga

SEQ ID NO: 500 - RNA, 17 nt, Homo sapiens
uaauugcuuccauguuu

SEQ ID NO: 501 - RNA, 51 nt, Homo sapiens
ucuguguaaaccuggcaauuuucacuuaauugcuuccauguuuauaaaaga